(19) United States
(12) Patent Application Publication
(10) Pub. No.: US 2025/0186516 A1
(43) Pub. Date: Jun. 12, 2025

(54) LIGILACTOBACILLUS PROBIOTICS, LIGILACTOBACILLUS EXTRACELLULAR VESICLES AND METHODS OF USING SAME

(71) Applicant: UNIVERSITY OF OTTAWA, Ottawa (CA)

(72) Inventors: Riadh HAMMAMI, Ottawa (CA); Saba MIRI, Ottawa (CA); Walid Abdelfattah Elsayed MOTTAWEA, Ottawa (CA)

(21) Appl. No.: 18/816,776

(22) Filed: Aug. 27, 2024

Related U.S. Application Data
(60) Provisional application No. 63/607,682, filed on Dec. 8, 2023.

(51) Int. Cl. A61K 35/742

(52) U.S. Cl. CPC A61K 35/742

(57) ABSTRACT

There is provided Ligilactobacillus probiotics, Ligilactobacillus extracellular vesicles and methods of using same. The methods include methods of preventing bacteria growth. Also provided are compositions.

### RELATED APPLICATIONS AND INCORPORATION BY REFERENCE

This application claims the benefit of priority from U.S. Provisional Application No. 63/607,682 filed Dec. 8, 2023. The disclosures of all of the above applications are incorporated by reference herein in their entireties.

### SEQUENCE LISTING

The instant application contains a sequence listing which has been submitted electronically in xml format and is hereby incorporated by reference in its entirety. Said xml copy, created on Aug. 8, 2024, is named 2372-115pr SEQ Listing and is 7,483 bytes in size.

### FIELD OF THE INVENTION

The present invention pertains to the field of preventing microbial infection or contamination and more particularly directed to Ligilactobacillus probiotics, Ligilactobacillus extracellular vesicles and methods of using same.

### BACKGROUND OF THE INVENTION

Many studies have demonstrated that lactic acid bacteria (LABs) are effective probiotics for inhibiting pathogenic microorganisms, degrading mycotoxins, and stimulating the immune system of the host (Chlebicz and Śliżewska, 2020; Hernández-González et al., 2021; Lone et al., 2021). Campylobacter and Salmonella are the most common pathogenic strains that cause foodborne illness. The World Health Organization (WHO) reported in 2018 that one out of every ten individuals experiences a food-borne illness every year (Abukhattab et al., 2022). Antibiotics can be an effective treatment for Salmonella enterica serovar Typhimurium and Campylobacter jejuni infections, but their use must be carefully considered to avoid contributing to the growing problem of antibiotic resistance (White et al., 2002). Probiotics have been suggested as an alternative to antibiotics for treatment of bacterial infections including those that cause foodborne illness. Anti-microbial characteristics of LABs against foodborne pathogens have been demonstrated (Lin et al., 2008; Lone et al., 2021). The use of LABs as a probiotic to prevent Salmonella spp. infection in poultry has been proposed.

In 2020, the International Journal of Systematic and Evolutionary Microbiology (IJSEM) proposed a reclassification of the Lactobacillus genus, and one of the changes included the creation of a new genus called Ligilactobacillus. This reclassification aimed to provide a more accurate representation of the phylogenetic relationships within the group.

The Ligilactobacillus genus of lactic acid bacteria is associated with vertebrate hosts, formed through the 2020 division of the Lactobacillus genus. Ligilactobacillus includes Ligilactobacillus acidipiscis, Ligilactobacillus agilis, Ligilactobacillus animalis, Ligilactobacillus apodemi, Ligilactobacillus araffinosus, Ligilactobacillus aviarius, Ligilactobacillus ceti, Ligilactobacillus equi, Ligilactobacillus faecis, Ligilactobacillus hayakitensis, Ligilactobacillus murinus, Ligilactobacillus pabuli, Ligilactobacillus pobuzihii, Ligilactobacillus ruminis, Ligilactobacillus saerimneri, Ligilactobacillus salitolerans, and Ligilactobacillus salivarius.

Ligilactobacillus salivarius includes probiotic strains shown to inhibit pathogen growth and stimulate host immune responses (Yang et al., Front Immunol. 2022; 13:1034727.) Bacterial extracellular vesicles (EVs) interact with both bacteria and host cells (Caruana and Walper, 2020). EVs can be located either in cell-free supernatants or cell-debris of bacteria. A few classes of EVs have been recently discovered, including migrasomes (500-3000 nm), oncosomes/large oncosomes (1000-10000 nm), exophers (3.5-4 μm); in addition to a non-membranous EVs called exomeres (<50 nm) Exomeres are non-membranous extracellular nanoparticles and considered the smallest subtypes of EVs with sizes less than 50 nm. Its recent discovery poses a myriad of questions as its biogenesis and functional properties have yet to be identified. In contrast to exomeres, exosomes are considered the most well-studied subtype of EVs, ranging from 30 to 150 nanometers. It originates from endosomes by inward budding of endosomal membrane forming early endosomes. These early endosomes mature into multivesicular bodies (MVBs), which contain intraluminal vesicles (ILVs). When MVBs fuse with the plasma membrane, ILVs released into the extracellular space as exosomes. Exosomes have been shown to enter the bloodstream and cross the blood brain barrier via transcellular transport. Many implications of exosomes involve antigen presentation, immune regulation, and the transfer of genetic material between cells. Particularly, microbiota derived exosomes have shown to influence immune response, intestinal barrier function, and metabolic homeostasis. On the contrary, microvesicles, also known as ectosomes or shedding vesicles as it directly shed from the cell surface. It is formed by the direct outward budding and fusion of the plasma membrane ranging in size from 100 to 1,000 nanometers carrying a cytoplasmic cargo (Heijnen et al. 1999).

Although initially identified in Gram-negative bacteria, Gram-positive bacteria which release EVs have been identified (Briaud and Carroll, 2020). In the past decade, there has been an increase in interest in Gram-positive EVs, especially from pathogenic bacteria and their cargo as well as their immune responses on host cell. For examples, EVs derived from Staphylococcus aureus have been found to trigger inflammation in cases of atopic dermatitis, while EVs from Bacillus anthracis were discovered to transport anthrax toxin. A few studies showed that LABs secret EVs with antimicrobial activity against pathogens. EVs originating from Lactobacillus acidophilus play an important role in controlling the growth of competing bacteria by carrying antimicrobial peptides such as bacteriocins (Dean et al., 2020). Research has shown that the EV from Lactobacillus johnsonii N6.2 carries RNA (da Silva et al., 2023). According to their findings, RNA transcripts within the EV largely represent those genes that are the most abundantly transcribed in bacterial cells, such as genes involved in protein synthesis and glycolysis. They showed EVs mediate RNA-dependent immune response on host cell (da Silva et al., 2023). Dominguez Rubio et al., (2022) reviewed the potential use of EVs from probiotics as carriers for therapy in bacterial infections.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present disclosure broadly provides Ligilactobacillus probiotics, Ligilactobacillus extracellular vesicles and methods of using same.

In an aspect of the present invention, there is provided a composition for preventing bacteria growth comprising Ligilactobacillus sp., including one or more of Ligilactobacillus salivarius, and Ligilactobacillus saerimneri or combination thereof and more specifically one or more of Ligilactobacillus salivarius UO.C109, Ligilactobacillus saerimneri UO.C121 and Ligilactobacillus salivarius UO.C249 or combination thereof.

In some embodiments, the compositions of the invention prevent growth of Gram-negative bacteria selected from the group consisting of Escherichia genus, Salmonella genus, Vibrio genus and Campylobacter genus.

In some embodiments, the composition further comprises a further active ingredient including extracellular vesicles isolated from Ligilactobacillus sp and/or further probiotic microorganisms and/or prebiotics. Optionally, the bacterial extracellular vesicles are isolated from Ligilactobacillus sp., preferably Ligilactobacillus salivarius, and Ligilactobacillus saerimneri or combination thereof, more preferably from Ligilactobacillus salivarius UO.C109, Ligilactobacillus saerimneri UO.C121 and Ligilactobacillus salivarius UO.C249 or combination thereof.

In some embodiments, the composition is a probiotic composition.

In some embodiments, the composition is a postbiotic composition.

In some embodiments, the composition is formulated as a therapeutic composition, an additive including food or cosmetic additive or animal feed. Optionally, in embodiments where the composition is a therapeutic composition, the therapeutic composition is formulated for oral or topical administration.

Optionally, in embodiments where the composition is formulated as an animal feed additive, it is formulated as a livestock feed additive or aquaculture food additive. In some embodiments, the livestock feed additive is a cattle feed additive, equine feed additive, porcine feed additive or poultry fee additive. In some embodiments, the feed additive further comprises prebiotics.

In some embodiments, the composition is formulated as a surface treatment or wash.

In some embodiments, the probiotic composition is for preventing Gram negative bacteria growth.

In some embodiments, the probiotic composition is for preventing Gram positive bacteria growth.

In another aspect of the invention, there is provided a method of treating and/or preventing food poisoning in a subject, the method comprising administering a therapeutically effective amount of the probiotic composition of the invention.

In another aspect of the invention, there is provided a method of improving intestinal health and/or reducing bacterial pathogen load in a subject, the method comprising administering to the subject an effective amount of composition of the invention.

In some embodiments of the methods of the invention, the subject is a mammal, optionally a human or domesticated animal, a fish optionally an aquiculture fish species, a bird, optionally a poultry, reptile or amphibian.

In another aspect of the invention, there is provided a food product comprising the composition of the invention.

In another aspect of the invention, there is provided an animal feed comprising the composition of the invention, optionally the animal feed is livestock feed or aquaculture feed.

In another aspect of the invention, there is provided a composition for preventing Gram-negative bacteria growth comprising bacterial extracellular vesicles isolated from Ligilactobacillus sp. including one or more of Ligilactobacillus salivarius, and Ligilactobacillus saerimneri or combination thereof and more specifically one or more of Ligilactobacillus salivarius UO.C109, Ligilactobacillus saerimneri UO.C121 and Ligilactobacillus salivarius UO.C249 or combination thereof. In some embodiments, the composition further comprises a further active ingredient including extracellular vesicles isolated from another Ligilactobacillus sp or other probiotic and/or further probiotic microorganisms and/or prebiotics and/or postbiotics. In some embodiments, the composition is formulated as a therapeutic composition, an additive including a food or cosmetic additive or animal feed. In some embodiments, the EV containing compositions are formulated as a surface treatment or wash. In some embodiments, the EV compositions are formulated as a pharmaceutical composition, optionally formulated for oral or topical administration.

In some embodiments, the EV compositions of the invention prevent growth of Gram-negative bacteria selected from the group consisting of Escherichia genus, Salmonella genus, Vibrio genus and Campylobacter genus.

In some embodiments, the EV compositions further comprise a second active ingredient, optionally wherein the second active ingredient is an antibiotic.

In another aspect of the invention, there is provided a method of treating and/or preventing food poisoning in a subject, the method comprising administering a therapeutically effective amount of the extracellular vesicle containing composition of the invention.

In another aspect of the invention, there is provided a food product comprising the extracellular vesicle containing composition of the invention.

In another aspect of the invention, there is provided an animal feed comprising the extracellular vesicle containing composition of the invention, optionally the animal feed is livestock feed or aquiculture feed.

In another aspect of the invention, there is provided a poultry feed comprising the extracellular vesicle containing composition of the invention.

In another aspect of the invention, there is provided a method of improving intestinal health and/or reducing bacterial pathogen load in a poultry animal, the method comprising administering to the poultry animal an effective amount of the extracellular vesicle containing composition of the invention.

In another aspect of the invention, there is provided a method of improving intestinal health and/or reducing bacterial pathogen load in livestock, the method comprising administering to the livestock an effective amount of the extracellular vesicle containing composition of the invention.

In another aspect of the invention, there is provided a livestock feed comprising the extracellular vesicle containing composition of the invention.

In another aspect of the invention, there is provided a method of inhibiting biofilm growth, the method comprising treating a surface with the extracellular vesicle containing composition of the invention.

In another aspect of the invention, there is provided a method of reducing bacterial pathogen load on a surface, the method comprising treating a surface with the extracellular vesicle containing composition of the invention. In some embodiments, the method is to reduce contamination of eggs, vegetables or other food surfaces. In other embodiments, the surface is a food preparation surface, food storage surface or a hard surface.

### BRIEF DESCRIPTION OF THE FIGURES

These and other features of the invention will become more apparent in the following detailed description in which reference is made to the appended drawings.

FIG. 1 illustrates the effect of dilution on the CFS inhibitory effect against S. Typhimurium. (A) CFS from Ligilactobacillus salivarius UO.C109, and (B) CFS from Ligilactobacillus saerimneri UO.C121.

FIG. 2 illustrates survival of selected strains under simulated chicken GIT conditions. Control: Simulated gastric juice at pH 7.

FIG. 3 illustrates post-treatment shifts in Enterobacteriaceae and Lactobacillaceae. (A) Changes in Enterobacteriaceae abundance over time in the control and test groups. (B) Changes in Lactobacillaceae abundance over time. Control−ve: Chicken cecal microbiota (control group); Probiotics: Chicken cecal microbiota supplemented with probiotics (probiotic control group); Control+ve: Chicken cecal microbiota infected with Salmonella typhimurium. Probiotics+S: Salmonella-infected chicken cecal microbiota treated with probiotics. nsP>0.05, *P<0.05 and **P≤0.01.

FIG. 4 illustrates shift in the chicken gut microbiota after Salmonella infection and probiotic treatment. (A) Changes in microbiome taxa after 6 h of treatment. (B) Changes in the microbiome taxa after 24 h of treatment. ControlN: Chicken cecal microbiota (control group), Probiotics: Chicken cecal microbiota supplemented with probiotics (probiotic control group), ControlP: Chicken cecal microbiota infected with Salmonella typhimurium. Probiotics+S: Salmonella-infected chicken cecal microbiota treated with probiotics.

FIG. 5. A-C illustrates alpha diversity of each sample calculated using Shannon indices at different times (6, 12, and 24 h) post-treatment; control-ve: chicken cecal microbiota obtained without any treatment (negative control). Control+ve: chicken cecal microbiota challenged with S. typhimurium. Probiotics: chicken cecal microbiota supplemented with the probiotic L. saerimneri UO.C121. Probiotics+S: chicken cecal microbiota mixed with S. typhimurium and L. saerimneri UO.C121 (treated group). D and E: beta-diversity using principal coordinate analysis of Bray-Curtis distances; samples are colored either by time post-treatment (D) or the treatment group (E). PERMANOVA was used to test microbiota similarity among the tested groups, and p-values are shown. ns P>0.05, *P<0.05 and **P≤0.01.

FIG. 6 illustrates quantification of Salmonella abundance in chicken gut microbiota, CH1+S and CH2+S: chicken gut microbiota from donors 1 and 2 challenged with S. Typhimurium. CH1+S+P and CH2+S+P: challenged chicken gut microbiota from donors 1 and 2 treated with probiotic strains. *p<0.05, **p<0.01, ***p<0.001, ****p<0.0001

FIG. 7 illustrates short chain fatty acid (SCFA) profile changes in chicken gut microbiota, A) Normalized SCFAs over time, B) Correlation of SCFAs and 16S rRNA gene sequence abundance at the genus level. CH1+S and CH2+S: chicken gut microbiota from donors 1 and 2 challenged with S. typhimurium. CH1+S+P and CH2+S+P: challenged chicken gut microbiota from donors 1 and 2 treated with the probiotic combination. *p<0.05, **p<0.01, ***p<0.001, ****p<0.0001.

FIG. 8 shows adhesion and invasion of S. typhimurium into Caco-2 cells in the presence of selected probiotic strains (Ligilactobacillus salivarius UO.C109 and Ligilactobacillus saerimneri UO.C121) in single and coculture modes. *p<0.05, **p<0.01, ***p<0.001, ****p<0.0001 as compared to challenged Caco-2 cells with S. Typhimurium of treated sample

FIG. 9 provides an overview of the extracellular vesicle (EV) isolation methods used in this study and protein concentration before and after purification of EVs.

FIG. 10 shows transmission electron microscopy image after the negative staining and inhibitory assay and total protein concentration of EVs in mixture and fraction 1 to fraction 3 (F1-F3). (A) EVs from Ligilactobacillus saerimneri UO.C121. (B) EVs from Ligilactobacillus salivarius UO.C109. (C) EVs from Ligilactobacillus salivarius UO.C249.

FIG. 11 details proteomics of EVs in mixture and fraction 1 to fraction 3 (F1-F3) obtained from selected probiotics. (A) Principal-component analysis plots for strains and their EVs, (B) Venn diagrams showing the number of identified proteins, (C) Molecular/biological Function and localization GO-term enrichment analysis, (D) Heat map of EVs from selected strains based on conserved protein domain.

FIG. 12 details peptidoglycan hydrolase activity in fraction 3 of selected stains (Ligilactobacillus saerimneri UO.C121, Ligilactobacillus salivarius UO.C109, Ligilactobacillus salivarius UO.C249). Control positive and negative contained lysozyme and PBS buffer in the peptidoglycan mixture respectively.

FIG. 13 illustrates particle size distribution and concentration of EVs from Ligilactobacillus saerimneri UO.C121. (A) mixture, (B) Fraction 1; F1, (C) Fraction 2; F2, (D) Fraction 3; F3, (E) Fraction 4; F4.

FIG. 14 illustrates results of inhibitory assay of EVs in mixture and fraction 1 to fraction 3 (F1-F3). A) EVs from Ligilactobacillus salivarius UO.C249 against S. typhimurium B) EVs from Ligilactobacillus saerimneri UO.C121 against Campylobacter jejuni C) EVs from EVs from Ligilactobacillus salivarius UO.C109.

FIG. 15 illustrates antimicrobial activity of EVs assessed through optical density and cell counting analysis (A) EVs from Ligilactobacillus saerimneri UO.C121 against S. Typhimurium. (B) EVs from Ligilactobacillus salivarius UO.C109 against S. Typhimurium. (C) EVs from Ligilactobacillus salivarius UO.C249 against Campylobacter jejuni. The negative control consists of medium with EVs but without bacteria, and the positive control includes bacteria in its respective medium. The symbol X denotes probiotic EVs without dilution. Diluted samples are represented as X/2, X/4, X/8, and X/16, indicating EVs with 2, 4, 8, and 16×times dilution factors, respectively. ns means P>0.05, * means P≤0.05, ** means P≤0.01, *** means P≤0.001, **** means P≤0.0001.

FIG. 16 shows relative expression of virulence genes in S. typhimurium and C. jejuni treated with EVs mixture and F3 compared to the control. (a) invA gene in S. Typhimurium, (b) SopE2 gene in S. typhimurium, (c) arvA gene in S. typhimurium, (d) hi/A gene in S. typhimurium, and (e) virulence genes in C. jejuni. ^{ns}P>0.05, *P≤0.05, **P≤0.01, ***P≤0.001, ****P≤0.0001.

### DETAILED DESCRIPTION OF THE INVENTION

The present invention relates to probiotic Ligilactobacillus sp, compositions comprising the same, and/or extracellular vesicles derived therefrom and there use as a bactericidal and/or bacteriostatic agent. Also provided are methods of preventing or inhibiting bacterial growth using Ligilactobacillus sp, compositions comprising the same, and/or extracellular vesicles derived therefrom.

### Definitions

Unless defined otherwise, all technical and scientific terms used herein have the same meaning as commonly understood by one of ordinary skill in the art to which this invention pertains.

As used herein, the term “extracellular vesicles” or “EV” means any vesicle of a lipid nature or membrane-enclosed structure, released spontaneously or induced (by culture conditions or by physicochemical treatments) in the extracellular environment (medium) by the probiotic, and containing at least one biologically active component produced by the probiotic including lipids, proteins, peptides including bacteriocins nucleic acids or exopolysaccharides. EVs play a crucial role in cell-to-cell communication by carrying various bioactive molecules, such as proteins, lipids, and nucleic acids (such as RNA and DNA). Extracellular vesicles can be classified into different types, including exosomes, microvesicles, and apoptotic bodies, based on their biogenesis and size.

As used herein, a substance is “pure” or “isolated” if it is substantially free of other components. The terms “purify,” “purifying” and “purified” refer to a EV or other material that has been separated from at least some of the components with which it was associated either when initially produced or generated (e.g., whether in nature or in an experimental setting), or during any time after its initial production. An EV may be considered purified if it is isolated at or after production, such as from one or more other bacterial components. In some embodiments, purified EVs are more than about 80%, about 85%, about 90%, about 91%, about 92%, about 93%, about 94%, about 95%, about 96%, about 97%, about 98%, about 99%, or more than about 99% pure. EV compositions and the microbial components thereof are, e.g., purified from residual habitat products.

The term “probiotic” means live microorganisms that, when administered in adequate amounts, confer a health benefit on the host. Probiotics include bacteria and yeast which confer health benefits when consumed in adequate amounts.

As used herein, the term “purified EV composition” or “EV composition” refer to a preparation that includes EVs that have been separated from at least one associated substance found in a source material (e.g. separated from at least one other bacterial component) or any material associated with the EVs in any process used to produce the preparation. It also refers to a composition that has been significantly enriched or concentrated. In some embodiments the EVs are concentrated by 2 fold, 3-fold, 4-fold, 5-fold, 10-fold, 100-fold, 1000-fold, 10,000-fold or more than 10,000 fold.

As used herein, the terms “subject” refers to any animal. A subject described as “in need thereof” refers to one in need of a treatment for a disease and/or reduction in pathogenic bacterial load. Mammals (i.e., mammalian animals) include humans, laboratory animals (e.g., primates, rats, mice), livestock (e.g., cows, sheep, goats, pigs), and household pets (e.g., dogs, cats, rodents). Subject also includes birds including poultry, reptiles and amphibians.

As used herein the term “poultry” relates to the class of domesticated fowl (birds) used for food or for their eggs. Poultry includes wildfowl, waterfowl, and game birds. Examples of poultry include, but are not limited to, chicken, broilers, bantams, turkey, duck, geese, guinea fowl, peafowl, quail, dove, pigeon (squab), and pheasant.

### Probiotic Bacteria

There is provided probiotic bacteria from Ligilactobacillus genus and in particular, Ligilactobacillus salivarius, and Ligilactobacillus saerimneri. In some embodiments, the probiotic bacteria are Ligilactobacillus salivarius UO.C109, Ligilactobacillus saerimneri UO.C121 and Ligilactobacillus salivarius UO.C249 or closely related strains. In some embodiments, probiotic bacteria from other genus are used in combination with the probiotic bacteria from Ligilactobacillus genus disclosed herein.

In some embodiments, the probiotic strain is the Ligilactobacillus salivarius UO.C109 deposited with International Depository Authority of Canada (IDAC) (National Microbiology Laboratory, Public Health Agency of Canada, 1015 Arlington Street, Winnipeg, Manitoba, Canada R3E 3R2) in its capacity as an International Depository Authority on Nov. 21, 2023 under Accession Number 211123-01.

In some embodiments, the probiotic strain is the Ligilactobacillus saerimneri UO.C121 deposited with International Depository Authority of Canada (IDAC) (National Microbiology Laboratory, Public Health Agency of Canada, 1015 Arlington Street, Winnipeg, Manitoba, Canada R3E 3R2) in its capacity as an International Depository Authority on Nov. 21, 2023 under Accession Number 211123-02.

In some embodiments, the probiotic strain is the Ligilactobacillus salivarius UO.C249 deposited with International Depository Authority of Canada (IDAC) (National Microbiology Laboratory, Public Health Agency of Canada, 1015 Arlington Street, Winnipeg, Manitoba, Canada R3E 3R2) in its capacity as an International Depository Authority on Nov. 21, 2023 under Accession Number 211123-03

In some embodiments, the probiotic strain is L. plantarum MTCC 1407 and/or L. acidophilus MTCC 10307 (IMTECH, Chandigarh, India) which can inhibit the E. coli ATCC 25922.

In some embodiments, the probiotic strain is Lactobacillus plantarum CCFM8610, L. plantarum C88, and/or L. plantarum strain 21B, which have strong antimicrobial activity against Escherichia coli and Salmonella.

In some embodiment, the probiotic strain is Lactobacillus acidophilus CICC 6074 and/or EPEC 086:K61 strains (China Center of Industrial Culture Collection) which inhibits Enteropathogenic Escherichia coli (EPEC).

In some embodiment, the probiotic strain is L. casei CRL 431 (CERELA culture collection) which have protection effect against Salmonella enteritidis serovar Typhimurium infection.

In some embodiments, the probiotic strain is L. rhamnosus JB3 (LR-JB3), isolated from a dairy product, interfered with the adhesion and invasion ability of H. pylori to AGS cells.

In some embodiments, the probiotic strain is a bacteriocin-producing strains of Lactobacillus plantarum inhibit adhesion of Staphylococcus aureus to extracellular matrix.

In some embodiments, the probiotic strain is a bacteriocin-like substance producing strain of Lactobacillus salivarius subsp. salivarius CRL 1328 with activity against Enterococcus faecalis, Enterococcus faecium, and Neisseria gonorrhoeae was characterized.

In some embodiments, the probiotic strain is Lactobacillus helveticus BGRA43, Lactobacillus fermentum BGHI14 or Streptococcus thermophilus BGVLJ1-44 which have inhibition effect against Clostridium difficile and Clostridium perfringens.

In some embodiments, the probiotic strain is Lactobacillus saerimneri strain GPV03, Lactobacillus saerimneri strain 20194, Lactobacillus saerimneri strain 10271, Lactobacillus saerimneri strain 1A, Lactobacillus saerimneri strain ClaCZ24, Lactobacillus saerimneri strain M-11, Lactobacillus saerimneri strain 13063, Lactobacillus saerimneri strain 9144, or Lactobacillus sp. T059 have been found to have around 100% coverage on 16S with our sequence (Lactobacillus saerimneri UO.C121).

In some embodiments, the probiotic bacteria has a 16s rRNA gene sequence at least 97%, 97.5%, 98%, 98.5%, 99%, 99.5%, 99.9% or 100% identical to the sequence set forth below:

(SEQ ID NO: 1)
GCAAGTCGAGCGCATCGGCCCAACTGATTGAAGATGCTTGCATCCGATTG
ACGATGGTTTACCGATGAGCGGCGGACGGGTGAGTAACACGTAGGTAACC
TGCCCAGAAGCGGGGGATAACACCTGGAAACAGATGCTAATACCGCATAG
GTCATTTGACCGCATGGTCAAATGATTAAAGATGGCTCTGCTATCACTTC
TGGATGGACCTGCGGCGTATTAGCTAGTTGGTAAGGTAACGGCTTACCAA
GGCAATGATACGTAGCCGAGTTGAGAGACTGATCGGCCACATTGGGACTG
AGACACGGCCCAGACTCCTACGGGAGGCAGCAGTAGGGAATCTTCCACAA
TGGACGCAAGTCTGATGGAGCAACGCCGCGTGAGCGAAGAAGGTCTTCGG
ATCGTAAAACTCTGTTGTTAGAGAAGAACACGGGTGAGAGTAACTGTTCA
CCTGTTGACGGTATCTAACCAGCAAGTCACGGCTAACTACGTGCCAGCAG
CCGCGGTAATACGTAGGTGGCAAGCGTTATCCGGATTTATTGGGCGTAAA
GGGAACGCAGGCGGTTCTTTAAGTCTGATGTGAAAGCCTTCGGCTTAACC
GAAGATGTGCATTGGAAACTGGGGAACTTGAGTGCAGAAGAGGAGAGTGG
AACTCCATGTGTAGCGGTGAAATGCGTAGATATATGGAAGAACACCAGTG
GCGAAAGCGGCTCTCTGGTCTGTAACTGACGCTGAGGTTCGAAAGCGTGG
GTAGCGAACAGGATTAGATACCCTGGTAGTCCACGCCGTAAACGATGAAT
GCTAGGTGTTGGAGGGTTTCCGCCCTTCAGTGCCGCAGCTAACGCACTAA
GCATTCCGCCTGGGGAGTACGACCGCAAGGTTGAAACTCAAAGGAATTGA
CGGGGGCCCGCACAAGCGGTGGAGCATGTGGTTTAATTCGAAGCTACGCG
AAGAACCTTACCAGGTCTTGACATCTTTTGACCACCTAAGAGATTAGGTT
TTCCCTTCGGGGACAAAATGACAGGTGGTGCATGGTTGTCGTCAGCTCGT
GTCGTGAGATGTTGGGTTAAGTCCCGCAACGAGCGCAACCCTTGTTGTCA
GTTGCCAGCATTCAGTTGGGCACTCTGGCGAGACTGCCGGTGACAAACCG
GAGGAAGGTGGGGATGACGTCAAATCATCATGCCCCTTATGACCTGGGCT
ACACACGTGCTACAATGGGCAGTACAACGAGTCGCGAAACCGCGAGGTTT
AGCAAATCTCTTAAAGCTGCTCTCAGTTCGGACTGCAGGCTGCAACTCGC
CTGCACGAAGTCGGAATCGCTAGTAATCGCGAATCAG

In some embodiments, the probiotic strain is Ligilactobacillus salivarius strain 665, Ligilactobacillus salivarius strain 1264, Ligilactobacillus salivarius strain 1068, Ligilactobacillus salivarius strain 1018, Ligilactobacillus salivarius strain 4782, Ligilactobacillus salivarius strain IBB3154, Ligilactobacillus salivarius strain PHENJ2, Ligilactobacillus salivarius strain DSM 103789, Ligilactobacillus salivarius strain LGM8-7, Ligilactobacillus salivarius strain BCRC 14759, or Ligilactobacillus salivarius strain ZLS006.

In some embodiments, the probiotic bacteria has a 16s rRNA gene sequence at least 97%, 97.5%, 98%, 98.5%, 99%, 99.5%, 99.9% or 100% identical to the sequence set forth below:

(SEQ ID NO: 2)
AACGAAACTTTCTTACACCGAATGCTTGCATTCACCGTAAGAAGTTGAGT
GGCGGACGGGTGAGTAACACGTGGGTAACCTGCCTAAAAGAAGGGGATAA
CACTTGGAAACAGGTGCTAATACCGTATATCTCTAAGGATCGCATGATCC
TTAGATGAAAGATGGTTCTGCTATCGCTTTTAGATGGACCCGCGGCGTAT
TAACTAGTTGGTGGGGTAACGGCCTACCAAGGTGATGATACGTAGCCGAA
CTGAGAGGTTGATCGGCCACATTGGGACTGAGACACGGCCCAAACTCCTA
CGGGAGGCAGCAGTAGGGAATCTTCCACAATGGACGCAAGTCTGATGGAG
CAACGCCGCGTGAGTGAAGAAGGTCTTCGGATCGTAAAACTCTGTTGTTA
GAGAAGAACACGAGTGAGAGTAACTGTTCATTCGATGACGGTATCTAACC
AGCAAGTCACGGCTAACTACGTGCCAGCAGCCGCGGTAATACGTAGGTGG
CAAGCGTTGTCCGGATTTATTGGGCGTAAAGGGAACGCAGGCGGTCTTTT
AAGTCTGATGTGAAAGCCTTCGGCTTAACCGGAGTAGTGCATTGGAAACT
GGAAGACTTGAGTGCAGAAGAGGAGAGTGGAACTCCATGTGTAGCGGTGA
AATGCGTAGATATATGGAAGAACACCAGTGGCGAAAGCGGCTCTCTGGTC
TGTAACTGACGCTGAGGTTCGAAAGCGTGGGTAGCAAACAGGATTAGATA
CCCTGGTAGTCCACGCCGTAAACGATGAATGCTAGGTGTTGGAGGGTTTC
CGCCCTTCAGTGCCGCAGCTAACGCAATAAGCATTCCGCCTGGGGAGTAC
GACCGCAAGGTTGAAACTCAAAGGAATTGACGGGGGCCCGCACAAGCGGT
GGAGCATGTGGTTTAATTCGAAGCAACGCGAAGAACCTTACCAGGTCTTG
ACATCCTTTGACCACCTAAGAGATTAGGTTTTCCCTTCGGGGACAAAGTG
ACAGGTGGTGCATGGCTGTCGTCAGCTCGTGTCGTGAGATGTTGGGTTAA
GTCCCGCAACGAGCGCAACCCTTGTTGTCAGTTGCCAGCATTAAGTTGGG
CACTCTGGCGAGACTGCCGGTGACAAACCGGAGGAAGGTGGGGACGACGT
CAAGTCATCATGCCCCTTATGACCTGGGCTACACACGTGCTACAATGGAC
GGTACAACGAGTCGCGAGACCGCGAGGTTTAGCTAATCTCTTAAAGCCGT
TCTCAGTTCGGATTGTAGGCTGCAACTCGCCTACATGAAGTCGGAATCGC
TAGTAATCGCGAA

In some embodiments, the probiotic strain is Ligilactobacillus salivarius strain 3173, Ligilactobacillus salivarius strain 1854, Ligilactobacillus salivarius strain 1733, Ligilactobacillus salivarius strain 1027, Ligilactobacillus salivarius strain 1006, Ligilactobacillus salivarius strain 6099, Ligilactobacillus salivarius strain 5579, Ligilactobacillus salivarius strain 5456, Ligilactobacillus salivarius strain 4789, Ligilactobacillus salivarius strain 8857, Ligilactobacillus salivarius strain 8029, Ligilactobacillus salivarius strain 6688, Ligilactobacillus salivarius strain L6, Ligilactobacillus salivarius strain BM 21, or Ligilactobacillus salivarius strain 17393.

In some embodiments, the probiotic bacteria has a 16s rRNA gene sequence at least 97%, 97.5%, 98%, 98.5%, 99%, 99.5%, 99.9% or 100% identical to the sequence set forth below:

(SEQ ID NO: 3)
TGCTATACATGCAAGTCGAACGAAACTTTCTTACACCGAATGCTTGCATT
CACCGTAAGAAGTTGAGTGGCGGACGGGTGAGTAACACGTGGGTAACCTG
CCTAAAAGAAGGGGATAACACTTGGAAACAGGTGCTAATACCGTATATCT
CTAAGGATCGCATGATCCTTAGATGAAAGATGGTTCTGCTATCGCTTTTA
GATGGACCCGCGGCGTATTAACTAGTTGGTGGGGTAACGGCCTACCAAGG
TGATGATACGTAGCCGAACTGAGAGGTTGATCGGCCACATTGGGACTGAG
ACACGGCCCAAACTCCTACGGGAGGCAGCAGTAGGGAATCTTCCACAATG
GACGCAAGTCTGATGGAGCAACGCCGCGTGAGTGAAGAAGGTCTTCGGAT
CGTAAAACTCTGTTGTTAGAGAAGAACACGAGTGAGAGTAACTGTTCATT
CGATGACGGTATCTAACCAGCAAGTCACGGCTAACTACGTGCCAGCAGCC
GCGGTAATACGTAGGTGGCAAGCGTTGTCCGGATTTATTGGGCGTAAAGG
GAACGCAGGCGGTCTTTTAAGTCTGATGTGAAAGCCTTCGGCTTAACCGG
AGTAGTGCATTGGAAACTGGAAGACTTGAGTGCAGAAGAGGAGAGTGGAA
CTCCATGTGTAGCGGTGAAATGCGTAGATATATGGAAGAACACCAGTGGC
GAAAGCGGCTCTCTGGTCTGTAACTGACGCTGAGGTTCGAAAGCGTGGGT
AGCAAACAGGATTAGATACCCTGGTAGTCCACGCCGTAAACGATGAATGC
TAGGTGTTGGAGGGTTTCCGCCCTTCAGTGCCGCAGCTAACGCAATAAGC
ATTCCGCCTGGGGAGTACGACCGCAAGGTTGAAACTCAAAGGAATTGACG
GGGGCCCGCACAAGCGGTGGAGCATGTGGTTTAATTCGAAGCAACGCGAA
GAACCTTACCAGGTCTTGACATCCTTTGACCACCTAAGAGATTAGGCTTT
CCCTTCGGGGACAAAGTGACAGGTGGTGCATGGCTGTCGTCAGCTCGTGT
CGTGAG

In other embodiments, the Ligilactobacillus genus probiotics are used in combination with one or more probiotic bacteria belonging to different genus or probiotic fungal strains. In some embodiments, one or more probiotic bacteria belonging to different genus are selected from the group consisting of Lactobacillus, Bifidobacterium, Streptococcus, and Lactococcus.

In other embodiments, the probiotic bacteria is a probiotic strain listed in the table below:

| Probiotic strains | Targeted pathogenic strains |
|---|---|
| Lactobacillus acidophilus | Escherichia coli (E. coli) |
| Lacticaseibacillus rhamnosus | Aggregatibacter |
| Lacticaseibacillus casei | actinomycetemcomitans |
| Lacticaseibacillus paracasei | Bacillus cereus |
| Lactiplantibacillus plantarum | Campylobacter jejuni |
| Levilactobacillus brevis | Candida albicans |
| Ligilactobacillus salivarius | Chlamydia trachomatis |
| Limosilactobacillus fermentum | Clostridium spp. |
| Limosilactobacillus reuteri | Clostridium difficile |
| Lactobacillus delbrueckii | Enterococcus faecalis |
| Lactobacillus crispatus | Gardnerella vaginalis |
| Lactobacillus gasseri | Helicobacter spp. |
| Lactobacillus johnsonii | Klebsiella spp. |
| Lactobacillus helveticus | Listeria monocytogenes |
| Lactobacillus sakei | Neisseria gonorrhoeae |
| | Pseudomonas spp. |
| | Prevotella |
| | Clostridium difficile |
| | Streptococcus mutans |
| | Staphylococcus aureus |
| | Salmonella |
| | Shigella |
| | Streptococcus mutans |
| | Pseudomonas aeruginosa |
| | Clostridium difficile |
| | Enterobacteriaceae |

In other embodiments, the probiotic bacteria is Lactobacillus acidophilus optionally for use against Escherichia coli (E. coli).

In other embodiments, the probiotic bacteria is Lactobacillus casei optionally for use against Salmonella.

In other embodiments, the probiotic bacteria is Lactobacillus rhamnosus optionally for use against Helicobacter pylori.

In other embodiments, the probiotic bacteria is Lactobacillus plantarum optionally for use against Staphylococcus aureus.

In other embodiments, the probiotic bacteria is Lactobacillus salivarius optionally for use against Enterococcus faecalis.

In other embodiments, the probiotic bacteria is Lactobacillus fermentum optionally for use against Clostridium difficile.

In other embodiments, the probiotic bacteria is Lactobacillus spp. optionally for use against Listeria monocytogenes.

In some embodiments, the probiotics are non-viable including heat-killed (including tyndallized) probiotic bacteria.

### Bacterial Derived Products

There is provided bacterial derived products including cell free supernatants and/or extracellular vesicles (EVs) from Ligilactobacillus and/or any probiotic bacteria disclosed herein. Optionally, the cell free supernatants and/or extracellular vesicles are derived from Ligilactobacillus salivarius, and Ligilactobacillus saerimneri. In preferred embodiments, the cell-supernatants and/or extracellular vesicles are from Ligilactobacillus salivarius UO.C109, Ligilactobacillus saerimneri UO.C121 and Ligilactobacillus salivarius UO.C249 or combination thereof.

In some embodiments, the cell free supernatants and/or extracellular vesicles (EVs) are derived Ligilactobacillus salivarius UO.C109 deposited with International Depository Authority of Canada (IDAC) (National Microbiology Laboratory, Public Health Agency of Canada, 1015 Arlington Street, Winnipeg, Manitoba, Canada R3E 3R2) in its capacity as an International Depository Authority on Nov. 21, 2023 under Accession Number 211123-01.

In some embodiments, the cell free supernatants and/or extracellular vesicles (EVs) are derived Ligilactobacillus saerimneri UO.C121 deposited with International Depository Authority of Canada (IDAC) (National Microbiology Laboratory, Public Health Agency of Canada, 1015 Arlington Street, Winnipeg, Manitoba, Canada R3E 3R2) in its capacity as an International Depository Authority on Nov. 21, 2023 under Accession Number 211123-02.

In some embodiments, the cell free supernatants and/or extracellular vesicles (EVs) are derived Ligilactobacillus salivarius UO.C249 deposited with International Depository Authority of Canada (IDAC) (National Microbiology Laboratory, Public Health Agency of Canada, 1015 Arlington Street, Winnipeg, Manitoba, Canada R3E 3R2) in its capacity as an International Depository Authority on Nov. 21, 2023 under Accession Number 211123-03.

In some embodiments, the EVs are between about 30 nm to 200 nm in diameter and have a visible lipid bilayer. In other embodiments, the EVs are between 80 nm to 90 nm in diameter.

Optionally, the EVs carry proteins, nucleic acids, and other molecules to target cells. They can fuse with the membranes of target bacteria, delivering their cargo directly into the bacterial cytoplasm.

In some embodiments, the EVs comprise one or more of peptidoglycan endopeptidase, a HAD superfamily hydrolase (metallopeptidase activity), and an ATP-dependent protease (threonine-type endopeptidase activity).

EVs can be produced according to any process known to those skilled in the art. The production process may for example comprise the following steps:
(a) cultivation of at least one bacterial strain under conditions suitable for the production of EVs,
(b) separating the at least one bacterial strain and the EVs produced in step (a) and,
(c) purification and concentration of EVs.

The culture step can be carried out under standard conditions known to those skilled in the art. For example, the culture step can be carried out in MRS/synthetic or agri-food residues-based media or basal media enriched with carbon or nitrogen sources, at 37° C. until late exponential or stationary phase is reached.

The separation and purification can also be carried out under standard conditions known to those skilled in the art. This may be, for example, a filtration step. For example, centrifugation can be carried out at 5000 g for 30 min and filtration can be carried out with a filter having a 100 kDa cut off point.

Some of the key methods and considerations for culturing bacteria for production of bioproducts such as EVs listed bellow:

Co-cultures and Mixed Cultures: The use of microbial co-cultures and mixed cultures has gained interest in bioproduct co-generation. Co-cultures involve the cultivation of two or more different microbial species, while mixed cultures encompass a more diverse microbial community.

Solid State Fermentation (SSF): SSF is a cultivation technique that has been used for the production of various bioproducts. It involves the growth of microorganisms on solid substrates in the absence or near absence of free water.

Submerge fermentation: Bioreactors are commonly used for the large-scale cultivation of microorganisms for bioproduct production in batch or continuous mode. This method is widely used for Lactobacillus strains at industrial scales for starter culture. The bacterial cells can be grown in free or immobilized form.

Microbial Consortia: The use of microbial consortia, including co-cultures of microalgae and bacteria, has been explored for the enhanced production of bioproducts. Understanding the interactions between different microorganisms can help optimize bioproduct yields.

Metabolic and Genetic Engineering: Metabolic and genetic engineering techniques can be employed to enhance the capabilities of microorganisms for bioproduct production. This may involve the manipulation of metabolic pathways, the introduction of novel genes, and the optimization of microbial strains for specific bioproducts.

The stabilization and preservation of EVs can be carried out under standard conditions known to those skilled in the art. For example, the stabilization and preservation step may include a drying and/or freezing step. In some embodiments, EVs are stabilized by lyophilization.

### Compositions

There are provided compositions including compositions comprising the bacteria from the genus Ligilactobacillus, including Ligilactobacillus salivarius, and Ligilactobacillus saerimneri and/or bacteria derived products from the genus Ligilactobacillus, including products derived from Ligilactobacillus salivarius, and Ligilactobacillus saerimneri. Bacterial derived products include EVs. The compositions may include different dosages of bacteria and/or EVs. In some embodiments, other probiotic microorganisms and/or products derived from other probiotic microorganisms and/or postbiotics are included.

In some embodiments, the compositions comprise a single bacterial strain from the genus Ligilactobacillus, including single strains of Ligilactobacillus salivarius, and Ligilactobacillus saerimneri, such as Ligilactobacillus salivarius UO.C109, Ligilactobacillus saerimneri UO.C121 and Ligilactobacillus salivarius UO.C249 or EVs isolated therefrom.

In other embodiments, the composition comprises two more different probiotic bacterial strains and/or EVs isolated from two or more different probiotic bacterial strains of the genus Ligilactobacillus, preferably Ligilactobacillus salivarius, and Ligilactobacillus saerimneri, including Ligilactobacillus salivarius UO.C109, Ligilactobacillus saerimneri UO.C121 and Ligilactobacillus salivarius UO.C249. In some embodiments, the composition Ligilactobacillus salivarius UO.C109, Ligilactobacillus saerimneri UO.C121 and Ligilactobacillus salivarius UO.C249 or EVs isolated therefrom.

In some embodiments, the composition comprises one or more probiotic bacterial strains the genus Ligilactobacillus and EVs isolated from one or more probiotic bacterial strains the genus Ligilactobacillus. Optionally, the probiotic bacteria are the same bacteria as the source of the EVs.

In some embodiments, the compositions include compositions consisting essentially of the bacteria the genus Ligilactobacillus. The compositions may include different dosages of the bacterium.

In some embodiments, the compositions comprise dead and/or inactive probiotic bacteria from the genus Ligilactobacillus.

In some embodiments, the compositions comprise heat-killed (including tyndallized) the bacteria, crude supernatant, partially purified EVs, crude culture, low growing microorganisms or components thereof. The formulation can consist of stabilizers such as cations, polysaccharides, polyphenols or proteins, or any combination of these compounds.

The compositions may be provided as a dried, powdered or lyophilized form.

The compositions may be provided as a solid form, immobilized on carriers, optionally oral form. Solid forms include tablets, capsules, pills, troches or lozenges, cachets, pellets, powders, or granules or incorporation of the material into particulate preparations of polymeric compounds such as polylactic acid, polyglycolic acid, etc. or into liposomes.

The compositions may be provided in liquid form including emulsions, solutions, suspensions, and syrups.

The compositions may include other components such as inert diluents; carriers, adjuvants, wetting agents, emulsifying and suspending agents; and sweetening, flavoring, and perfuming agents. optionally with an appropriate carrier or stabilizer.

In some embodiments, the composition comprising EVs further comprises one or more buffers, polymers including poloxamer 188 and/or sugar including sucrose. (See Enhancing the Stabilization Potential of Lyophilization for Extracellular Vesicles—Trenkenschuh—2022—Advanced Healthcare Materials—Wiley Online Library)

In some embodiments, the compositions are probiotic compositions.

In probiotic embodiments, other probiotic microorganisms including other lactic acid bacteria, including lactic acid bacteria selected from the group consisting of Lactobacillus, Bifidobacterium, Streptococcus and Lactococcus or combinations thereof may be included in the compositions. Optionally, the probiotic compositions also include probiotic fungal strains.

In some embodiments, the compositions include nutritional compositions, i.e., food products that comprise the probiotic bacteria or EVs alone or in combination with other probiotic bacteria. The food product can be a dairy product, for example, milk or a milk-based product. Exemplary milk sources include, without limitation, cattle, sheep, goat, yak, water buffalo, horse, donkey, reindeer and camel.

The milk can be whole milk or milk that has been processed to remove some or all of the butterfat, e.g., 2% milk, 1% milk or no-fat milk. In some embodiments, the milk can be previously pasteurized and or homogenized, dried and reconstituted, condensed or evaporated. Fractions of milk products including casein, whey protein or lactose may also be used.

The food product can be a cereal product, for example, rice, wheat, oats, barley, corn, rye, sorghum, millet, or triticale. The cereal product can be a whole grain or be milled into a flour. The food product can be a single kind of cereal or a mixture of two or more kinds of cereals, e.g., oat flour plus malted barley flour. The cereal products can be of a grade and type suitable for human consumption or can be products suitable for consumption by domestic animals.

The food product can also be a vegetable or a fruit product, for example, a juice, a puree, a concentrate, a paste, a sauce, a pickle or a ketchup. Exemplary vegetables and fruits include, without limitation, squashes, e.g., zucchini, yellow squash, winter squash, pumpkin, potatoes, asparagus, broccoli, Brussels sprouts, beans, e.g., green beans, wax beans, lima beans, fava beans, soy beans, cabbage, carrots, cauliflower, cucumbers, kohlrabi, leeks, scallions, onions, sugar peas, English peas, peppers, turnips, rutabagas, tomatoes, apples, pears, peaches, plums, strawberries, raspberries, blackberries, blueberries, lingonberries, boysenberries, gooseberries, grapes, currants, oranges, lemons, grapefruit, bananas, mangos, kiwi fruit, and carambola.

The food product can also be a “milk” made from grains (barley, oat or spelt “milk”) tree nuts (almond, cashew, coconut, hazelnut or walnut “milk”), legumes (soy, peanut, pea or lupin “milk”) or seeds (quinoa, sesame seed or sunflower seed “milk”).

Also contemplated are food products comprising animal proteins, for example, meat, for example, sausages, dried meats, fish and dried fish products and/or convenience foods.

The compositions include animal feed compositions including animal feed supplements and animal feed, i.e., feed products that comprise the probiotic bacteria or EVs alone or in combination with other probiotic bacteria.

In animal feed additive embodiments, the composition optional further comprises one or more vitamins, minerals, amino acids, and other necessary components. Compound feed may further comprise premixes.

Optionally, the animal feed additive or feed composition is for laboratory animals (e.g., primates, rats, mice), livestock (e.g., cows, sheep, goats, pigs), equines (including horses and donkeys), and household pets (e.g., dogs, cats, rodents). Animal feed and feed additives also includes feed and feed additives suitable for birds including poultry, reptiles, amphibians and fish including farmed species.

Compositions comprising EVs may be formulated as a composition to reduce or prevent bacterial contamination of surfaces including hard surfaces or food surfaces and optionally may be provided as an additive to reduce risk of bacterial contamination of household or consumer products including makeup or hygiene products.

In some embodiments, compositions comprising EVs may be used to reduce biofilm formation and/or surface contamination. In such embodiments, the composition may be formulated as a spray or foam or wash.

In some embodiments, the composition comprising EVs is for storage at −80° C.

In other embodiments, the composition comprising EVs is lyophilized.

### Methods

The disclosed probiotic bacteria and/or bacterial products including EVs and compositions comprising the same can be used, for example, to treat and/or preventing food poisoning in a subject. Optionally, the methods are used to treat or prevent food poisoning caused by Salmonella typhimurium or Campylobacter jejuni.

In some embodiments, Ligilactobacillus salivarius UO.C109 (L. S UO.C109), Ligilactobacillus saerimneri UO.C121 (L. saer UO.C121) bacteria, EVs or combination thereof are used in methods to prevent food poisoning caused by Salmonella typhimurium.

In other embodiments, Ligilactobacillus salivarius UO.C249 (L. S UO.C249) bacteria, EVs or combination thereof is used in methods to prevent food poisoning caused by Campylobacter jejuni.

In other embodiments, the disclosed probiotic bacteria and/or bacterial products including EVs and compositions comprising the same are used to improve gastrointestinal health and/or reducing bacterial pathogen load and/or virulence in a subject. Subjects include mammals, fish, birds, reptiles or amphibians. Optionally, the disclosed probiotic bacteria and/or bacterial products including EVs are provided as a food additive or part of the feed. The disclosed probiotic bacteria and/or bacterial products including EVs are optionally feed at least daily, weekly, or monthly. Optionally, the disclosed probiotic bacteria and/or bacterial products including EVs is provided shortly after birth, hatching or weaning.

The methods of the invention include methods of improving intestinal health and/or reducing bacterial pathogen load and/or virulence of farmed animals including poultry and farmed fish. In such embodiments, the disclosed probiotic bacteria and/or bacterial products including EVs is provided within 24 hr of hatching.

In some embodiments, the EVs of the invention can be used in methods of preventing or inhibiting bacterial growth in food products, including raw poultry meat, liquid eggs, milk, etc.

In some embodiments, the EVs of the invention can be used in methods of preventing or inhibiting bacterial growth including biofilm growth on surface including hard surfaces. Optionally EV containing compositions are useful in methods of reducing contamination of eggs, vegetables or other food surfaces and/or a food preparation surface, food storage surface.

To gain a better understanding of the invention described herein, the following examples are set forth. It will be understood that these examples are intended to describe illustrative embodiments of the invention and are not intended to limit the scope of the invention in any way.

### EXAMPLES

### Example 1: Bacteriocinogenic Ligilactobacillus Strain Association Inhibits Growth, Adhesion, and Invasion of Salmonella in a Simulated Chicken Gut Environment

A protective probiotic coculture to inhibit the growth of Salmonella enterica serovar Typhimurium in the simulated chicken gut environment was developed. Bacterial strains were isolated from the digestive mucosa of broilers and screened in vitro against Salmonella typhimurium ATCC 14028. A biocompatibility coculture test was performed, which identified two biocompatible strains, Ligilactobacillus salivarius UO.C109 and Ligilactobacillus saerimneri UO.C121 with high inhibitory activity against Salmonella. The cell-free supernatant (CFS) of the selected isolates exhibited dose-dependent effects, and the inhibitory agents were confirmed to be proteinaceous by enzymatic and thermal treatments. Proteome and genome analyses revealed the presence of known bacteriocins in the CFS of L. salivarius UO.C109, but unknown for L. saerimneri UO.C121. The addition of these selected probiotic candidates altered the bacterial community structure, increased the diversity of the chicken gut microbiota challenged with Salmonella, and significantly reduced the abundances of Enterobacteriaceae, Parasutterella, Phascolarctobacterium, Enterococcus, and Megamonas. It also modulated microbiome production of short-chain fatty acids (SCFAs) with increased levels of acetic and propionic acids after 12 and 24 hours of incubation compared to the microbiome challenged with S. typhimurium. Furthermore, the selected probiotic candidates reduced the adhesion and invasion of Salmonella to Caco-2 cells by 37-39% and 51%, respectively, after 3 h of incubation, compared to the control. These results suggest that the developed coculture probiotic strains has protective activity and could be an effective strategy to control Salmonella infections in poultry.

### Isolation and Screening of Bacteriocinogenic Strains

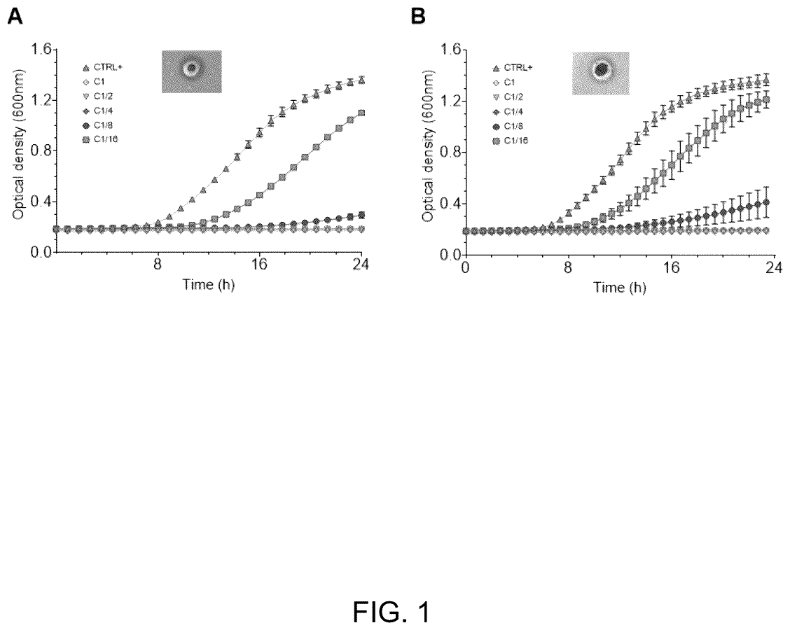

Of 290 colonies isolated from four intestinal tracts obtained from a local slaughterhouse, 22 strains demonstrated activity against S. typhimurium. As shown in Table 1, these isolates were identified as Ligilactobacillus salivarius (n=14), Lactobacillus johnsonii (n=6), Ligilactobacillus saerimneri (n=1), and Bacillus licheniformis (n=1). Table 1 summarizes the inhibitory activity of CFS produced by each isolate before and after proteolytic enzyme and thermal treatments. For instance, L. salivarius UO.C109 and L. saerimneri UO.C121 were sensitive to protease treatment, with no residual inhibition (dropping from 10 to 0 mm). After thermal treatment, the results indicated that some supernatants maintained their antimicrobial activity even after being exposed to treatment for 30 min at 100° C. For example, CFS from L. salivarius UO.C110 exhibited the highest thermal stability after 1 h of 100° C. treatment. In contrast, CFS from L. salivarius UO.C109 and L. saerimneri UO.C121 retained their activity for up to 30 min (8 mm of the inhibitory zone) but were completely inactivated after 1 h of treatment. CFS of the selected strains was serially diluted and assessed against S. enterica. As presented in FIG. 1, the CFSs of L. salivarius UO.C109 and L. saerimneri UO.C121 induced dose-dependent inhibition of S. Typhimurium over 24 h of incubation. The 8-fold dilution of CFS completely inhibited Salmonella growth, whereas a 16-fold dilution induced partial inhibition.

TABLE 1
Anti-Salmonella activity of selected strains and effect of
enzymatic and heat treatments on their cell-free supernatants

| | | Enzymatic | | | | |
|---|---|---|---|---|---|---|
| | | treatment | | Heat treatment (100° C.) | | |
| | | Inhibition | | Inhibition zone (mm) | | |
| | | zone (mm) | | 10 | 30 | 60 |
| Strains | Identifier | Before | After | min | min | min |
| Ligilactobacillus | UO.C101 | 10 | 8 | 10 | 10 | 8 |
| salivarius | | | | | | |
| L. salivarius | UO.C102 | 11 | 6 | 10 | 10 | 8 |
| L. salivarius | UO.C103 | 7 | 0 | nd | Nd | nd |
| L. salivarius | UO.C104 | 10 | 7 | 6 | 0 | 0 |
| L. salivarius | UO.C105 | 10 | 6 | 8 | 7 | 7 |
| L. salivarius | UO.C106 | 10 | 6 | 8 | 6 | 0 |
| L. salivarius | UO.C107 | 11 | 6 | 9 | 0 | 0 |
| L. salivarius | UO.C108 | 10 | 0 | 7 | 0 | 0 |
| L. salivarius | UO.C109 | 10 | 0 | 9 | 8 | 0 |
| L. salivarius | UO.C110 | 10 | 6 | 10 | 10 | 10 |
| L. salivarius | UO.C111 | 11 | 6 | 7 | 0 | 0 |
| L. salivarius | UO.C112 | 8 | 0 | 0 | 0 | 0 |
| L. salivarius | UO.C113 | 8 | 0 | nd | nd | nd |
| L. salivarius | UO.C114 | 8 | 0 | nd | nd | nd |
| L. johnsonii | UO.C115 | 7 | 0 | nd | nd | nd |
| L. johnsonii | UO.C116 | 8 | 0 | nd | nd | nd |
| L. johnsonii | UO.C117 | 8 | 0 | nd | nd | nd |
| L. johnsonii | UO.C118 | 7 | 0 | nd | nd | nd |
| L. johnsonii | UO.C119 | 7 | 0 | nd | nd | nd |
| L. johnsonii | UO.C120 | 7 | 0 | nd | nd | nd |
| L. saerimneri | UO.C121 | 10 | 0 | 9 | 9 | 0 |
| Bacillus | UO.C122 | 11 | 7 | 10 | 8 | 8 |
| licheniformis | | | | | | |

### Biocompatibility Test

A biocompatibility test was conducted to select the most biocompatible combination of the bioprotective strains. OD₂₄ (the optical density after 24 h), T_{lag} (the time when the optical density indicates an increase of 0.01 units) as well and Tₘₐₓ (the time when the optical density reaches its maximum) are presented in Tables 2 and S2. Table S2 shows that strains L. saerimneri UO.C121 and L. salivarius UO.C109 were compatible. L. salivarius UO.C109 had a lower lag phase in the presence of CFS than L. saerimneri UO.C121 (Table 2). However, the OD₂₄ of L. saerimneri UO.C121 reached 1.20, higher than that of the control (1.13).

TABLE 2
The growth characteristics of the selected strains
inoculated in the presence of CFSs and the
control group were cultured without adding CFS.

| | | Growth characteristics* | | | |
|---|---|---|---|---|---|
| | Inoculated | | | | Taux |
| MRS medium | strains | Tₘₐₓ (h) | T _{lag} (h) | OD ₂₄ | (OD/h) |
| Ligilactobacillus | Control | 4.23 | 0.98 | 1.99 | 0.2852 |
| salivarius | L. saerimneri | 3.31 | 0.68 | 2.20 | 0.3849 |
| UO.C109 | UO.C121 | | | | |
| Ligilactobacillus | Control | 4.33 | 0.59 | 1.13 | 0.0693 |
| saerimneri | L. salivarius | 3.97 | 1.08 | 1.20 | 0.0840 |
| UO.C121 | UO.C109 | | | | |

T_{lag} is the time when the optical density indicates an increase of 0.01 units, Tₘₐₓ is the time when the optical density reaches the maximum, OD₂₄ is the optical density after 24 h, and Tₐᵤₓ is the increase rate of the optical density per time.

### Survival of Probiotic Candidates in Simulated Gastrointestinal (GIT) Conditions

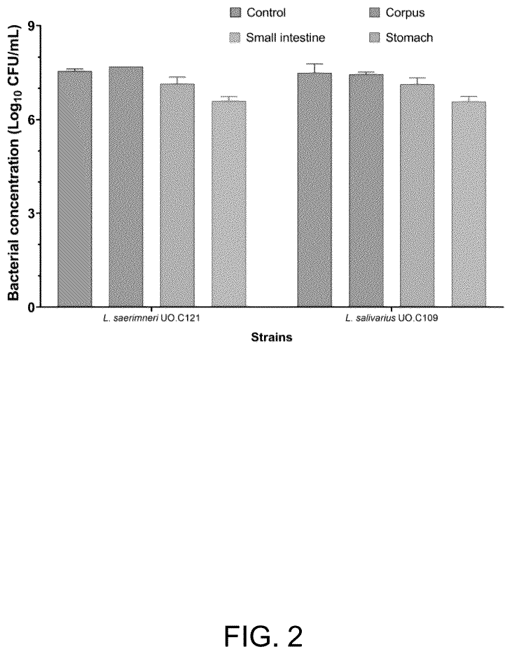

The selected probiotic candidates, L. salivarius UO.C109 and L. saerimneri UO.C121, showed strong tolerance to simulated gastrointestinal (GIT) conditions, and their time in the corpus (pH 5.4, 30 min), stomach (pH 2.8, 45 min), and small intestine (pH 6.1, 60 min) (FIG. 2). Despite a slight decrease in cell concentration after 1 h of incubation under small intestine conditions, both strains L. salivarius UO.C109 and L. saerimneri UO.C121 had a high survival rate with a respective range of 92-99% and 92-100% (FIG. 2).

### Genomic and Proteomic Profiling of the Selected Probiotic Candidates

### Whole Genome Sequencing and Bacteriocin Analysis

The length of the genome sequence, average G+C content, operons of the rRNA gene, and assembled contigs are summarized in Table 3. Genomic sequences of the selected strains were investigated to detect the presence of bacteriocin-encoding genes. Five bacteriocin genes were found in L. salivarius UO.C109, including Blp family class II bacteriocin and bacteriocin immunity protein. However, no known bacteriocin gene was detected in the genome of L. saerimneri UO.C121.

TABLE 3
An overview of genomic features of selected probiotic candidates

| Bacterial Identifier | UO.C109 | UO.C121 |
|---|---|---|
| Organism identity | Ligilactobacillus salivarius | Ligilactobacillus |
| | | saerimneri |
| NCBI Accession | JAPSFH000000000 | JAPTHF010000001 |
| Genome length (bp) | 1,909,609 | 1,661,176 |
| Genes (Total) | 1,955 | 1,657 |
| CDSs (total) | 1,867 | 1,585 |
| CDSs (with protein) | 1,833 | 1,567 |
| Number of RNAs | 88 | 72 |
| rRNAs | 4, 7, 7 (5S, 16S, 23S) | 2, 4, 1 (5S, 16S, 23S) |
| tRNAs | 66 | 61 |
| Identified bacteriocin | Bacteriocin | ND |
| genes | (MCZ0743448.1) | |
| | Bacteriocin | |
| | (MCZ0743449.1) | |
| | Blp family class II | |
| | bacteriocin | |
| | (MCZ0743450.1) | |
| | Blp family class II | |
| | bacteriocin | |
| | (MCZ0743451.1) | |
| | Bacteriocin | |
| | (MCZ0743452.1) | |
| Identified protease | 15 | 13 |
| genes | | |
| Identified hydrolase | 50 | 49 |
| genes | | |

ND: Not Determined

### Protein Profile of CFS

A total of 92 proteins were identified in the proteome of CFS from L. salivarius UO.C109 and L. saerimneri UO.C121 (Table S4). After annotation using the UniProt database, proteomic analysis revealed the presence of 30 and 32 unique proteins in CFS from L. salivarius UO.C109 and L. saerimneri UO.C121, respectively. In addition, a bacteriocin peptide (Abp118) was identified in L. salivarius UO.C109, whereas no known bacteriocin peptide was detected in the CFS of L. saerimneri UO.C121. Notably, both strains secreted in their CFS a peptidoglycan endopeptidase, a HAD superfamily hydrolase (metallopeptidase activity), and an ATP-dependent protease (threonine-type endopeptidase activity).

### Production of Organic Acids

The major SCFAs (acetic, propionic, and butyric acids) were quantified in the candidate probiotics using GC. Acetic acid was the main SCFA identified in the CFS, ranging from 24 to 27 mM for both probiotic candidates.

Impact on Chicken Gut Microbiota in the Presence or Absence of S. typhimurium

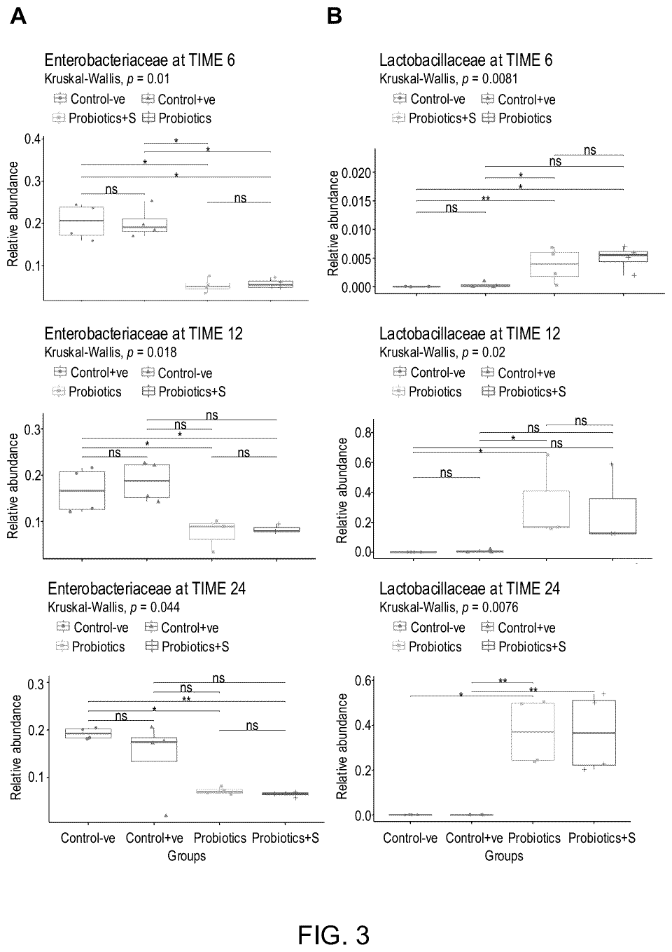

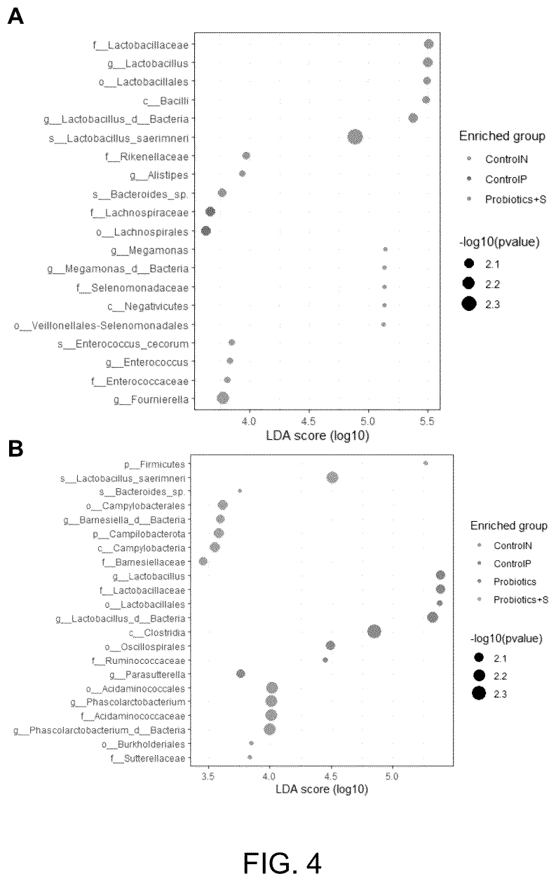

A 16S rRNA gene sequence analysis was performed to determine the structure of the bacterial communities. The results showed that Firmicutes (47.1%), Bacteroidetes (22.4%), Proteobacteria (19%), and Fusobacteria (1.3%) were the predominant taxonomic phyla in the chicken gut microbiota (Figure S2). Our results showed that there was significant compositional depletion in the predominant Enterobacteriaceae in the groups treated with probiotic strains with or without Salmonella as compared to the control group (Control−ve) and the group challenged with Salmonella (Control+ve) (FIG. 3A). Salmonella, Shigella, and E. coli are among the most common members of the Enterobacteriaceae family. Enrichment of Lactobacillaceae over time was confirmed in both groups (FIG. 3B). Changes in the taxonomic genera confirmed that the probiotic coculture shifted the fecal microbiome into a distinct taxonomic genus structure by decreasing the abundance of Parasutterella, Phascolarctobacterium, Enterococcus, and Megamonas (FIG. 4).

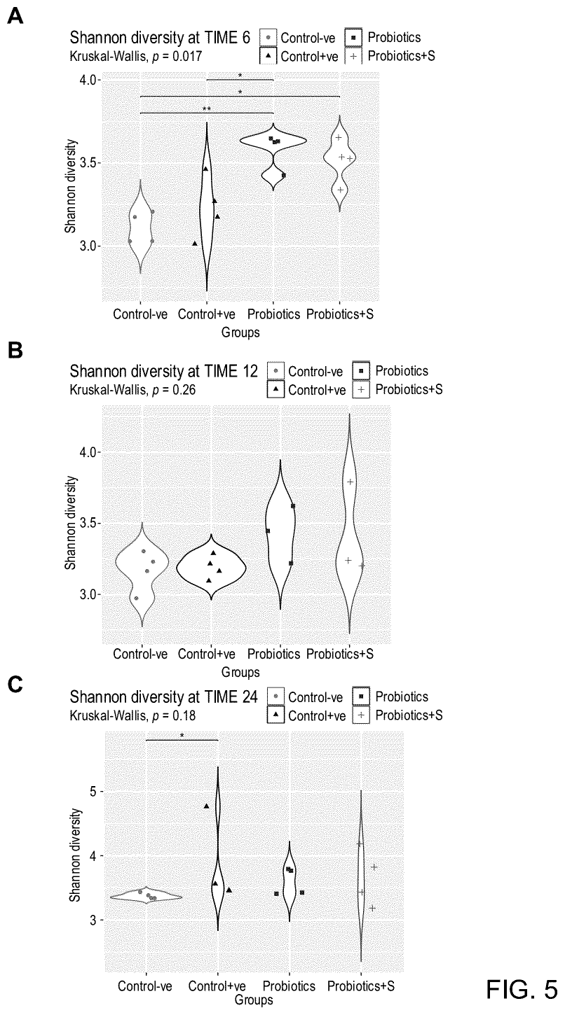

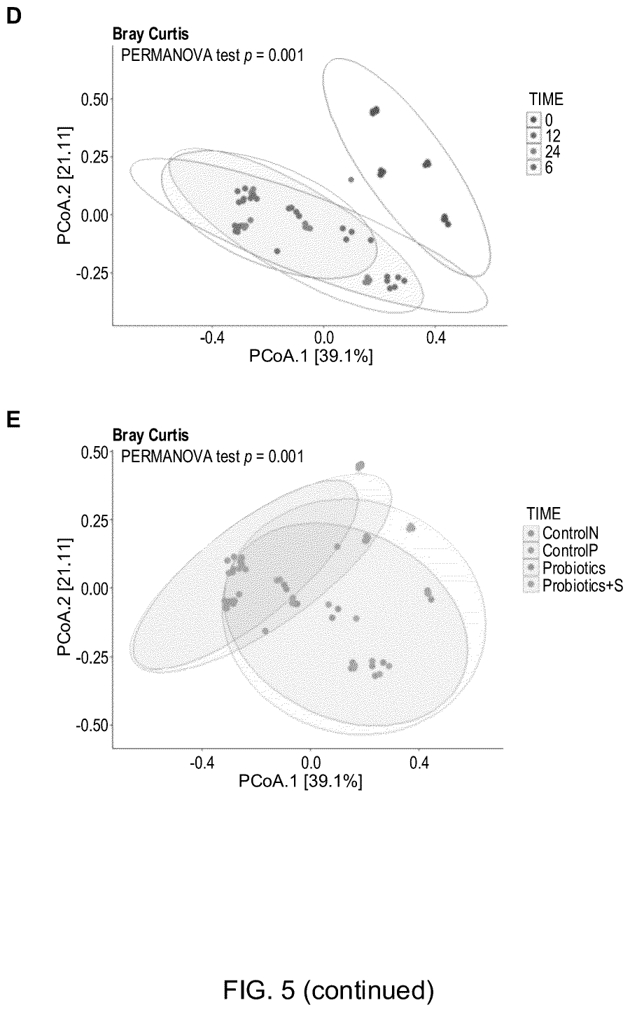

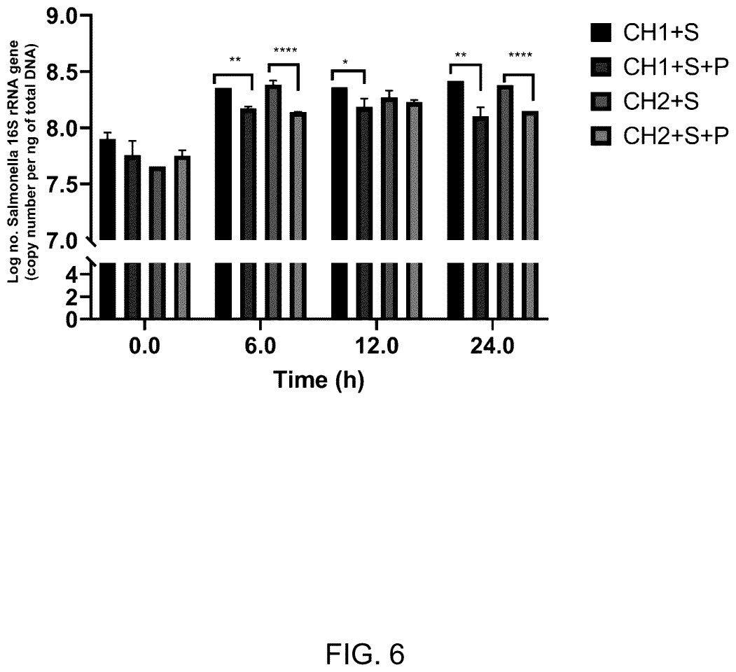

When alpha diversity indices were applied across all samples from different donors, Shannon indices were significantly higher in the two probiotic-treated groups after 6 h of treatment, and no statistically significant differences were observed after 12 and 24 h (FIG. 5A-C). Beta diversity analysis was used to determine whether the microbial communities were similar or dissimilar between the samples. FIG. 6 illustrates beta diversity based on the Bray-Curtis distance matrix. The results showed that the microbiota of different treatments was similar at zero time, then started to diverge over time based on the treatment (FIG. 5D). Additionally, the chicken gut microbiome without any treatment (ControlN) and that challenged with S. typhimurium (ControlP) were different (P=0.001) from those treated with the selected probiotic strains with or without Salmonella (FIG. 5E).

The abundance of S. typhimurium levels in the simulated chicken gut was quantified using qPCR. The average copy number of the 16S rRNA gene copy was 7.5×10⁷ and 6.3×10⁷ for the microbiota challenged with S. typhimurium and probiotic-treated microbiota groups at time 0. The 16S rRNA copy number increased after 6 h of incubation (FIG. 6). The results showed that Salmonella abundance decreased significantly after 6 h of incubation in the presence of selected probiotic strains. As shown in FIG. 6, the results from 24 h confirmed the decrease in S. typhimurium 16S rRNA in the presence of selected probiotic strains. These results corroborate those obtained from the 16S metagenomic analysis of gut composition at the taxonomic level (FIG. 4).

### The Effect of the Selected Probiotic Combination on Gut Metabolite Production

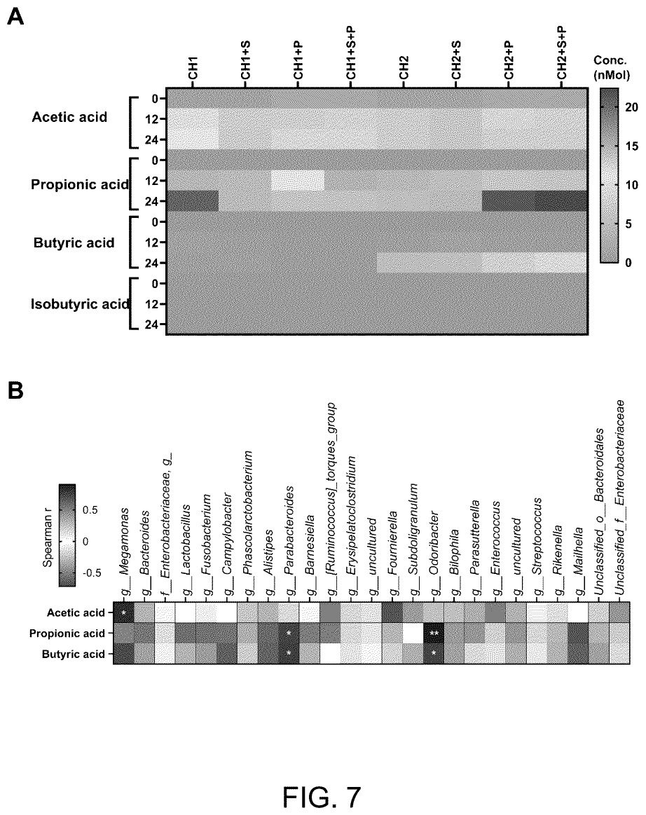

The SCFA profiles, including acetic acid, propionic acid, butyric acid, and isobutyric acid, were evaluated and summarized in FIG. 7A. Our results showed increased acetic and propionic acid levels in all groups after 12 h of incubation. The control group without any treatment (CH1 and CH2) and the chicken gut microbiota supplemented with the probiotic combination (CH+P and CH+S+P) had higher concentrations of propionic acid than the gut microbiome challenged with S. typhimurium (CH1+S and CH2+S). To assess the association between the gut microbiota and SCFAs concentrations, a correlation analysis was performed using Spearman's correlation coefficients for the 16S rRNA gene sequence and SCFAs levels at 24 h (FIG. 7B). The results indicated that acetic, propionic, and butyric acids were significantly associated with Megamonas, Parabacteroides, and Odoribacter (p<0.05). In addition, propionic and butyric acids were positively correlated with Lactobacillus spp.

Probiotic Inhibition of the Adhesion and Invasion of S. typhimurium to Epithelial Cells

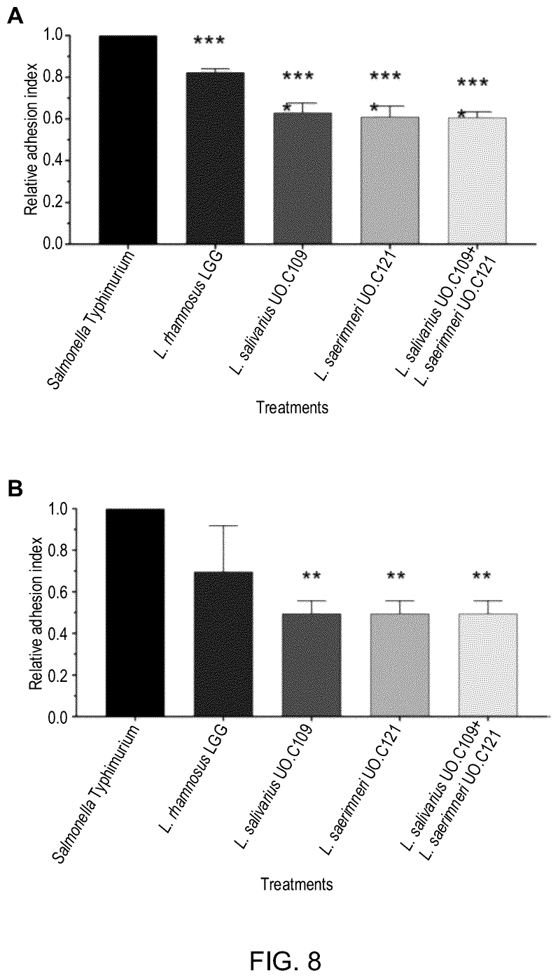

The adhesion and invasion of undifferentiated Caco-2 cells by S. typhimurium are shown in FIG. 8. L. rhamnosus LGG, a well-known protective probiotic strain, was used as a positive control. Single and cocultures (ratio 1:1) of selected probiotic candidates at a concentration of 10⁹ CFU/mL against 10⁹ CFU/mL of S. typhimurium ATCC 14028. L. salivarius UO.C109, L. saerimneri UO.C121, and their coculture reduced Salmonella adhesion to Caco-2 cells by 37.1, 38.9, and 39.4%, respectively. Moreover, the selected probiotic strains reduced Salmonella invasion of Caco-2 cells by 51% in both single cultures and cocultures (FIG. 8B).

### Example 2: Pathogen Control Using Extracellular Vesicles from Ligilactobacillus

The size, shape, protein content, and antimicrobial effects of extracellular vesicles (EVs) secreted by three Gram-positive gut symbiont strains—Ligilactobacillus salivarius UO.C109 (L. S UO.C109), Ligilactobacillus saerimneri UO.C121 (L. Saer UO.C121) and Ligilactobacillus salivarius UO.C249 (L. S UO.C249) were characterized. EVs from L. S UO.C109 and L. Saer UO.C121 had inhibitory activity against Salmonella typhimurium while EVs from L. S UO.C249 inhibited the growth of Campylobacter jejuni.

### Results

### Comparison of Extracellular Vesicle (EVs) Isolation Methods

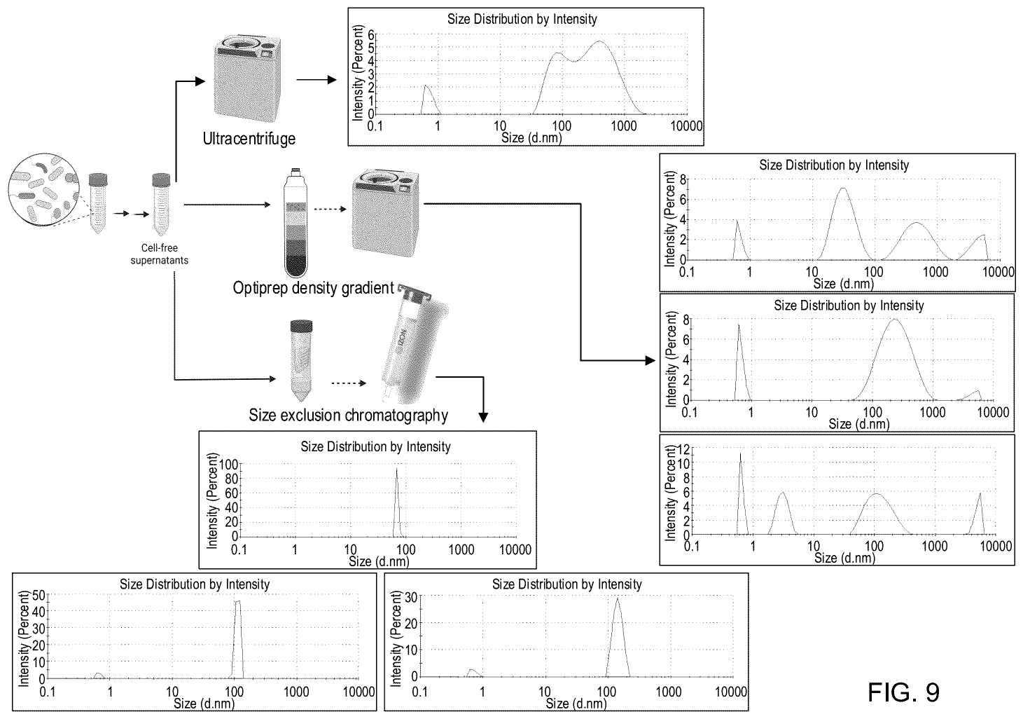

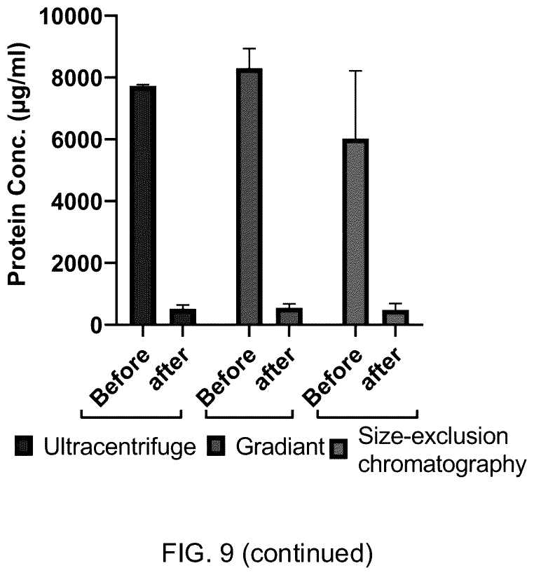

In order to determine whether selected probiotic strains produced EVs, UO.C109, UO.C121 and UO.C249 strains were cultured in De Man-Rogosa-Sharpe (MRS) medium for 72 h to reach stationary phase. EVs were isolated from cell-free supernatants using ultracentrifuge, OptiPrep™ density gradient (iodixanol (60% w/v), a density of 1.320±0.001 g/mL) and size exclusion chromatography. The quality of each method was evaluated by size distribution analysis and total protein concentration. All three methods, including OptiPrep™ density gradient, ultracentrifugation, and size exclusion chromatography, decreased the total protein content of samples up to 20-folds. This finding suggests that all three methods are effective in reducing the protein content of samples, which is important for the isolation and purification of EVs. However, the sized distribution in samples with the ultracentrifuge showed that wide nanosize particles were detected that were larger than the expected size of EVs, indicating the presence of some contaminating proteins in the samples (FIG. 9). On the other hand, there were some unexpected peaks in the purified samples using the OptiPrep™ density gradient method, suggesting the presence of remaining resin after purification (FIG. 9). This finding suggests that the OptiPrep™ density gradient method may not be the most effective method for purifying extracellular vesicles (EVs) from probiotic bacteria. Referring to the FIG. 9, the results from size exclusion chromatography provided three different EVs population (>100 nm, ˜100 nm, <100).

### Identification of Antimicrobial Properties and General Characterization of EVs Population

In this study, the differences in subpopulations of EVs were assessed by using a mixture of EVs and each fraction for TEM microscopy and antimicrobial tests. The TEM results demonstrated that the EVs were heterogeneous in size and had a spherical or cup-shaped morphology. The microscopy analysis revealed that the size of the EVs ranged between 30 and 200 nanometers and they had a visible lipid bilayer (FIG. 9). We found that the size of EVs in fraction 1 was significantly different from the size in the other fractions (FIG. 9). However, the size of EVs observed in the TEM images may be smaller than the expected size due to the drying process, which can cause dehydration of the EVs (Van Niel et al., 2018).

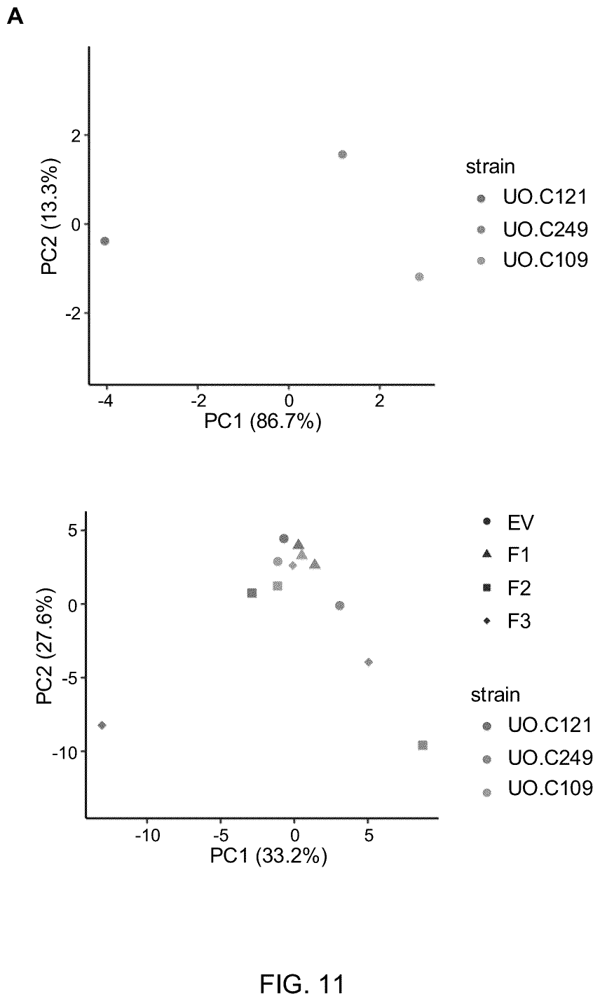

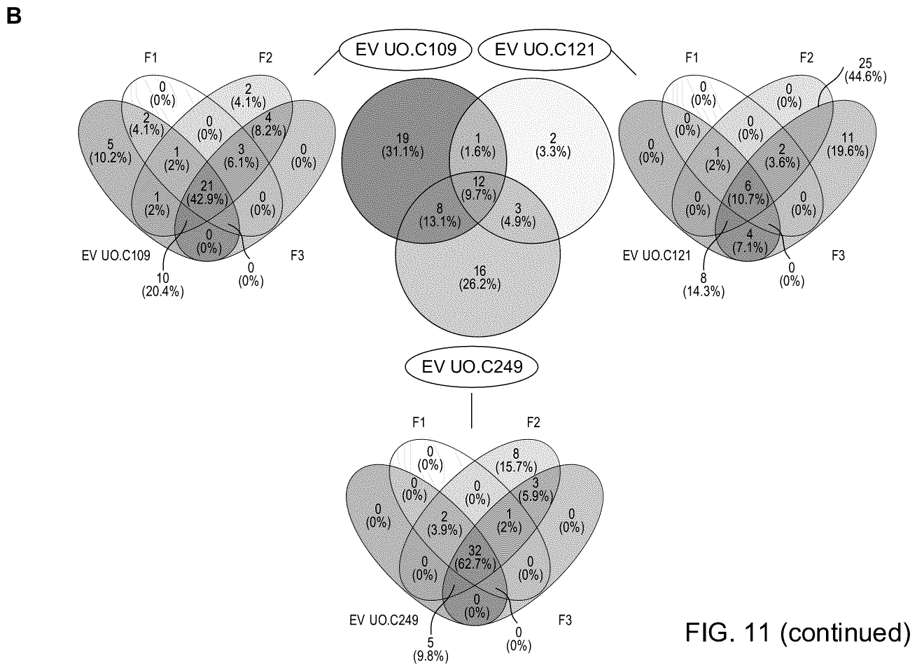

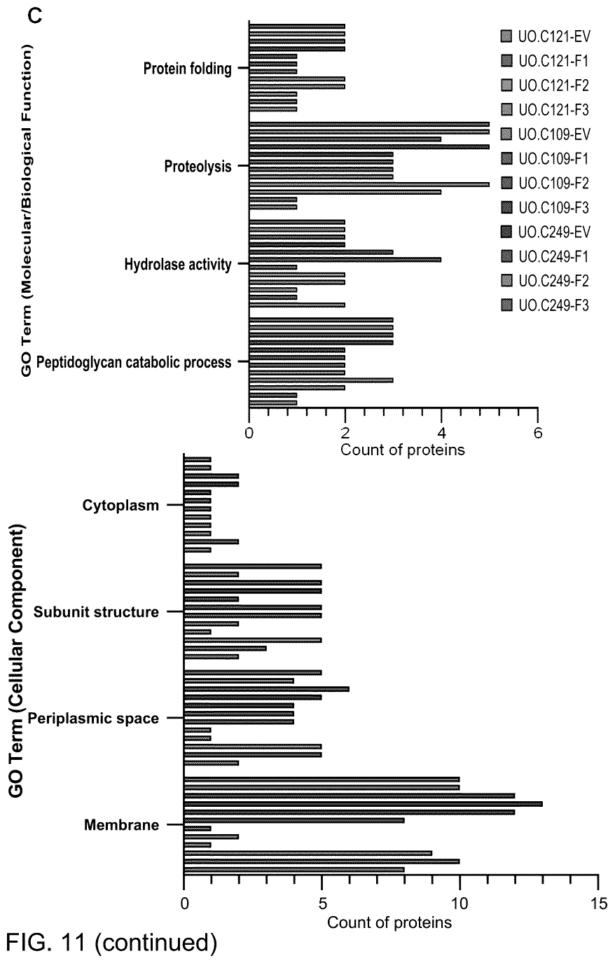

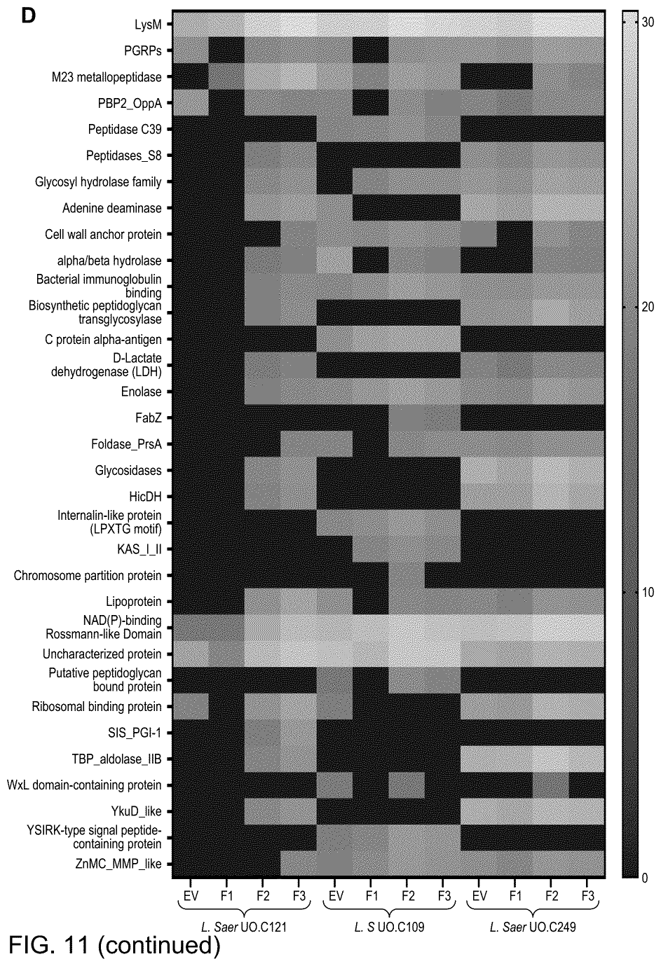

Each fraction is enriched with specific range size and protein content of this fraction is also enriched with some specific enzymes and proteins (FIG. 11). F1 (6.7E+9 particles per milliliter, 43.5% of 94.5 nanometers), F2 (2.5E+10 particles per milliliter, 58.8% of 85.8 nanometers), F3 (1.5E+10 particles per milliliter, 73.8% of 88.6 nanometers), F4 (5.7E+9 particles per milliliter, 40.2% of 81.7 nms), F3 had the highest concentration of EVs with 80-90 nm in size.

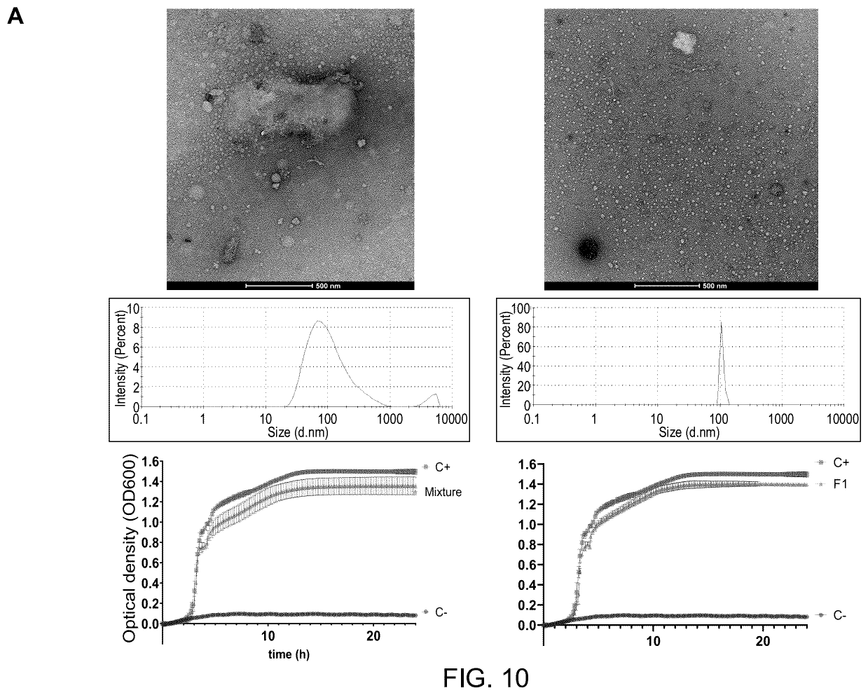

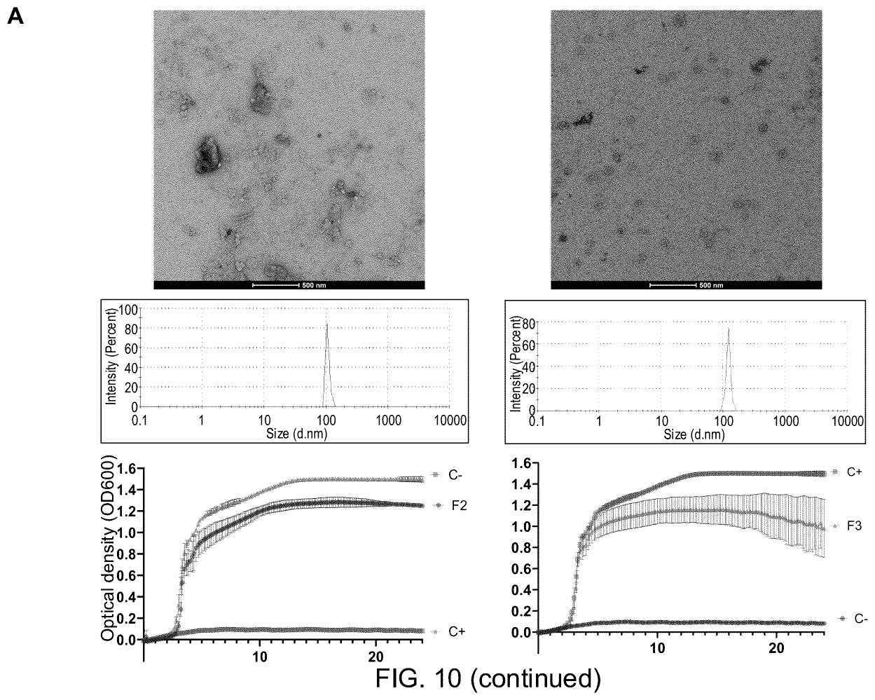

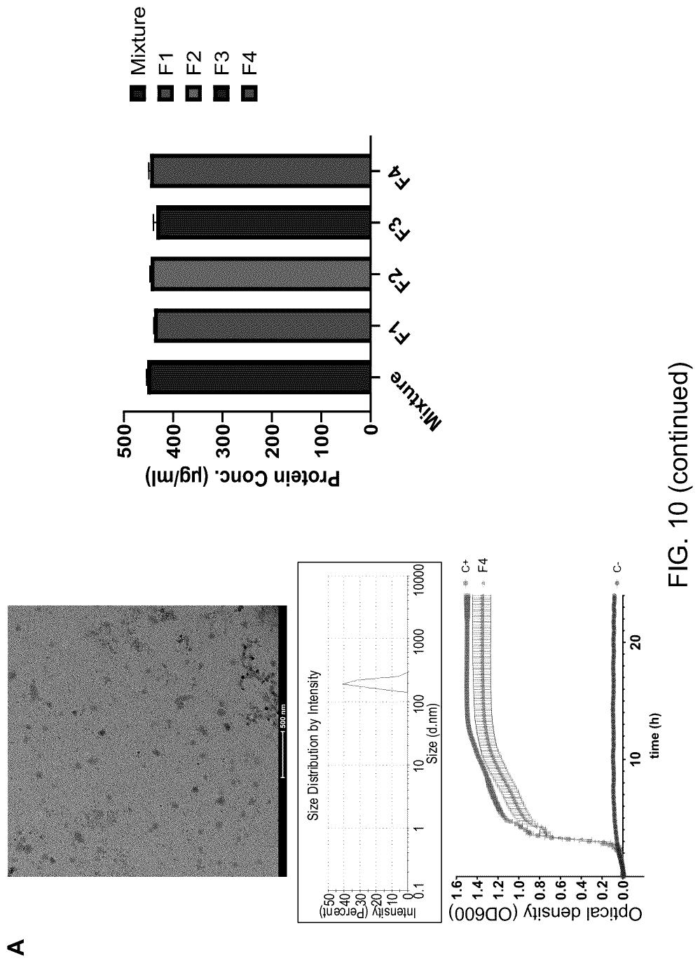

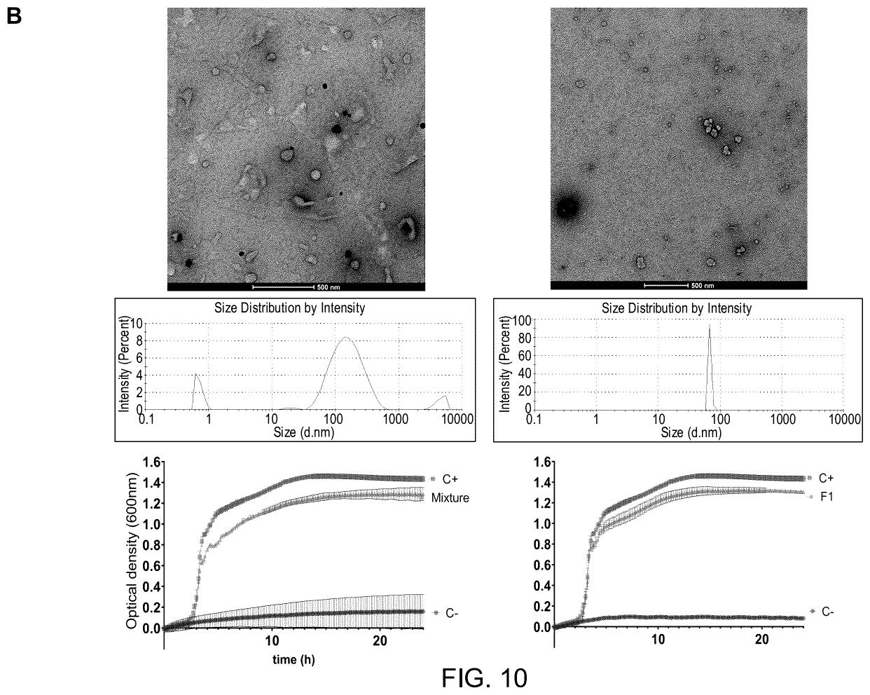

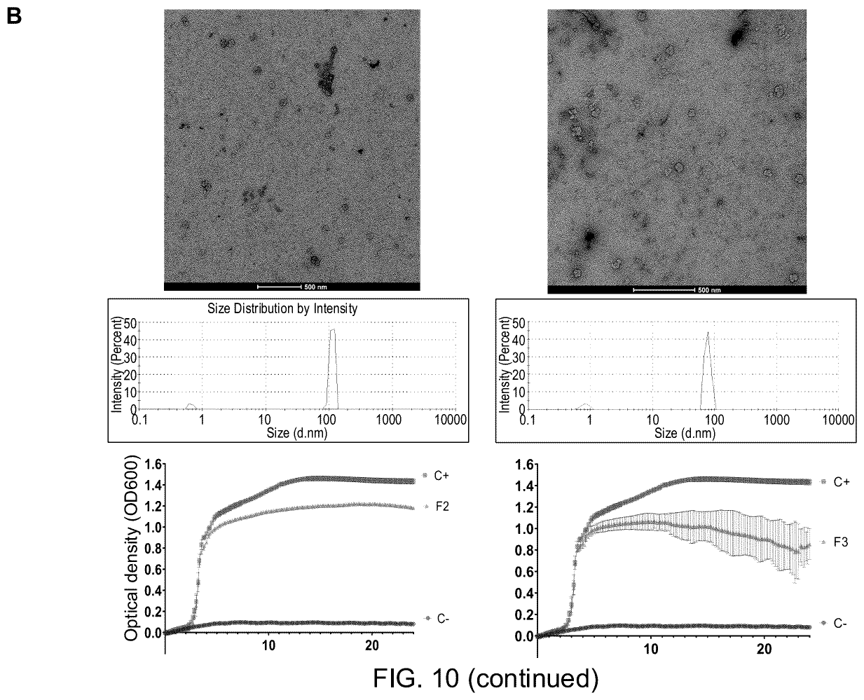

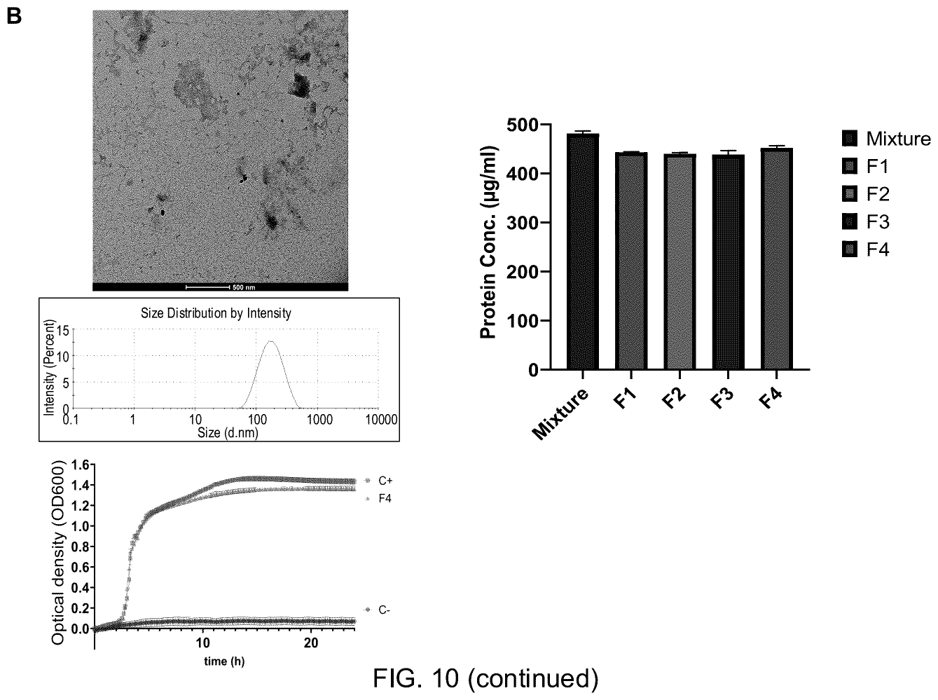

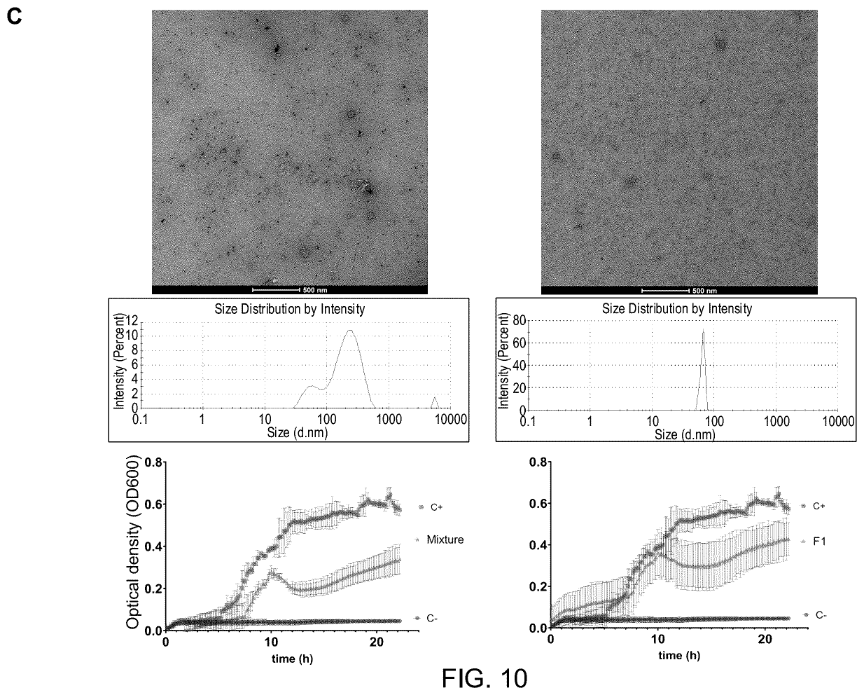

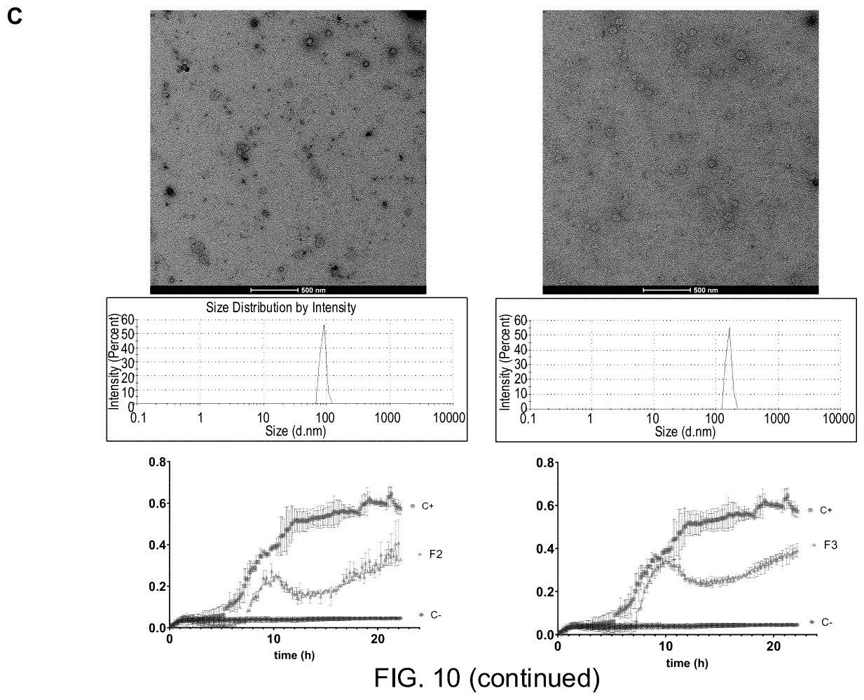

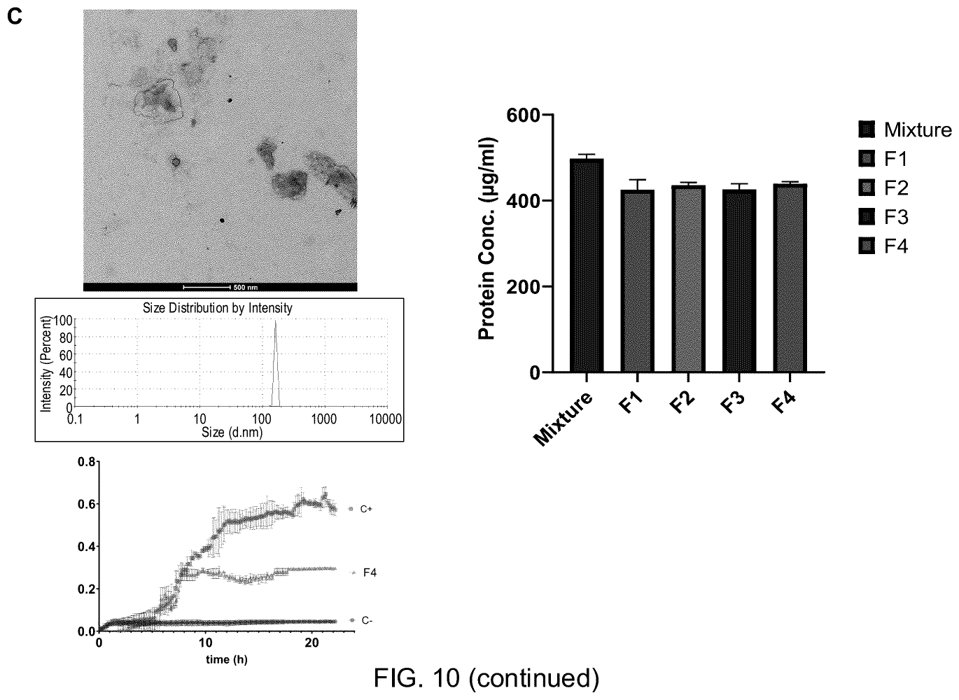

The study found that the EV fractions exhibited varying antimicrobial activity that was specific to their target pathogens. EVs derived from L. S UO.C109 and L. Saer UO.C121 showed inhibitory activity against S. typhimurium (FIGS. 10A and 10B), but not against Campylobacter jejuni. Purified fractions (F1-F4) were evaluated, and F3 was found to be the most active fraction with higher inhibitory activity compared to the EV mixture. The results also indicated that the antimicrobial activity of EVs could be enhanced through purification. However, F4 was not considered as a purified fraction due to the presence of protein contamination from supernatant. Thus, proteomic analysis was conducted on the EV mixture and F1 to F3. Additionally, EVs derived from L. S UO.C249 exhibited antimicrobial activity against only Campylobacter jejuni (FIG. 10C), but not against S. typhimurium. The results showed that F2 was found to be the most active fraction compared to the EV mixture, and F4 was not considered the most active fraction due to the presence of protein contamination. NTA concentration analysis on L. Saer UO.C121 revealed that the EV mixture contained 1.0E+10 particles per ml and 56.8% of those particles were 86.9 nanometers in size. Compared to F1 (6.7E+9 particles per milliliter, 43.5% of 94.5 nanometers), F2 (2.5E+10 particles per milliliter, 58.8% of 85.8 nanometers), F3 (1.5E+10 particles per milliliter, 73.8% of 88.6 nanometers), F4 (5.7E+9 particles per milliliter, 40.2% of 81.7 nms), F3 had the highest concentration of EVs with 80-90 nm in size.

### Proteomic Profile Analysis of EVs

To identify EVs protein cargo, quantitative mass spectrometry was used for the analysis of EVs mixtures and fractions 1 to 3. After filtering out contaminants and proteins with fewer than two mapped peptides, we found 18 proteins in EVs mixture from L. Saer UO.C121, 40 in the EVs mixture L. S UO.C109, and 39 in EVs mixture L. S UO.C249. Proteins present in only one replicate of any EV or fraction sample were considered missing in that sample. The Venn diagram of identified proteins showed that the EVs mixture from Ligilactobacillus salivarius UO.C109 and UO.C249 had 16 and 19 unique proteins respectively while only 2 unique proteins were identified in EVs mixture from Ligilactobacillus saerimneri UO.C121 (FIG. 11B). As shown in FIG. 11A, EVs from three selected strains displayed a very different PCA pattern. Also, F3 of EVs contained more identified proteins than F1, F2 and EVs mixture (FIG. 11B). While the majority of proteins detected in all fractions were found in the mixture of extracellular vesicles (EVs), a few proteins were not detected in the EV mixture (see FIG. 10B). This discrepancy could be attributed to certain proteins being highly abundant in the EV mixture. This makes it difficult for the mass spectrometer to detect low-abundance peptides. Consequently, a greater number of proteins were identified in the fractions.

Fusion mass spectrometer used had a peptide identification resolution of 60000.

KEGG pathway analysis showed that only 8 proteins were mapped and described as pathways. However, Gene Ontology (GO) analysis revealed that most of the detected proteins are located in the membrane and involved in protein and peptidoglycan degradation (FIG. 11C). The F3 fraction of EVs in all selected strains represented the highest abundance of membrane proteins and enzymes, which may be corroborated by the higher inhibitory activity of these fractions against pathogens. Family and domain databases predicted the function of proteins based on the conserved protein domain family (CCD database). As shown in FIG. 11D, most of the identified proteins with conserved domains that are involved in the degradation of bacterial cell walls. Our results showed that the lysin motif (LysM) had a significantly high abundance in all EVs samples (FIG. 11). This motif is involved in peptidoglycan binding and most of the identified LysM-containing proteins are peptidoglycan hydrolases that degrade bacterial cell walls. Also, it is noteworthy that LysM and other peptidoglycan binding motifs were found in antimicrobial peptides including bacteriocins (Martin-Visscher et al., 2009). Previously, we detected bacteriocins from cell-free supernatants of selected probiotics, including predicted bacteriocins in class III and Abp118, a class IIb bacteriocin (ref.). These results show the presence of bacteriocins in EVs as well. Peptidoglycan recognition proteins (PGRPs) were also detected in most of EVs samples except F1 from L. Saer UO.C121 and L. S UO.C109. PGRPs are pattern recognition receptors that bind to, and in certain cases, hydrolyze peptidoglycans (PGNs) of bacterial cell walls. This family includes Zn-dependent N-Acetylmuramoyl-L-alanine Amidase, EC:3.5.1.28. This enzyme cleaves the amide bond between N-acetylmuramoyl and L-amino acids, preferentially D-lactyl-L-Ala, in bacterial cell walls (Dziarski and Gupta, 2006). In addition, M23 family metallopeptidase, also known as beta-lytic metallopeptidase, was detected as a protein family of zinc endopeptidases that lyse bacterial cell wall peptidoglycans. The oligopeptide ABC transporter protein (Oppa) was also detected which is responsible for the uptake of a variety of substrates or ligands as a part of the ABC-type oligopeptide import system. The interaction of this oligopeptide with its substrate triggers ligand translocation across the cytoplasmic membrane energized by ATP hydrolysis (Nakayama et al., 2015). Besides transport proteins, the PBP2 superfamily includes ligand-binding domains from ionotropic glutamate receptors, LysR-type transcriptional regulators, and unorthodox sensor proteins involved in signal transduction (Tam and Saier Jr, 1993).

### Peptidoglycan Hydrolase

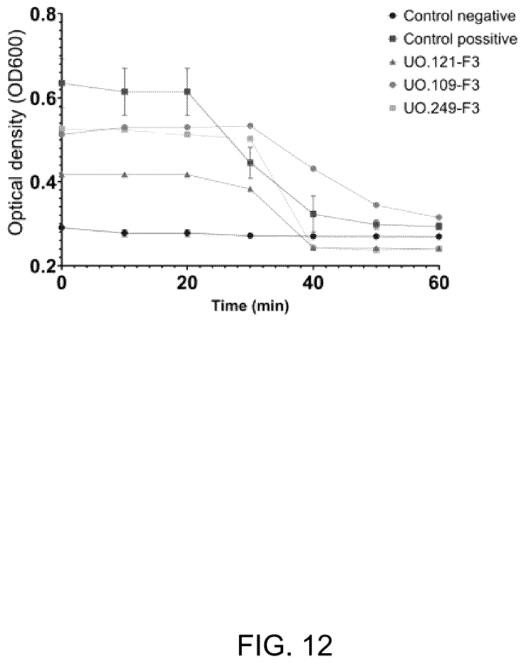

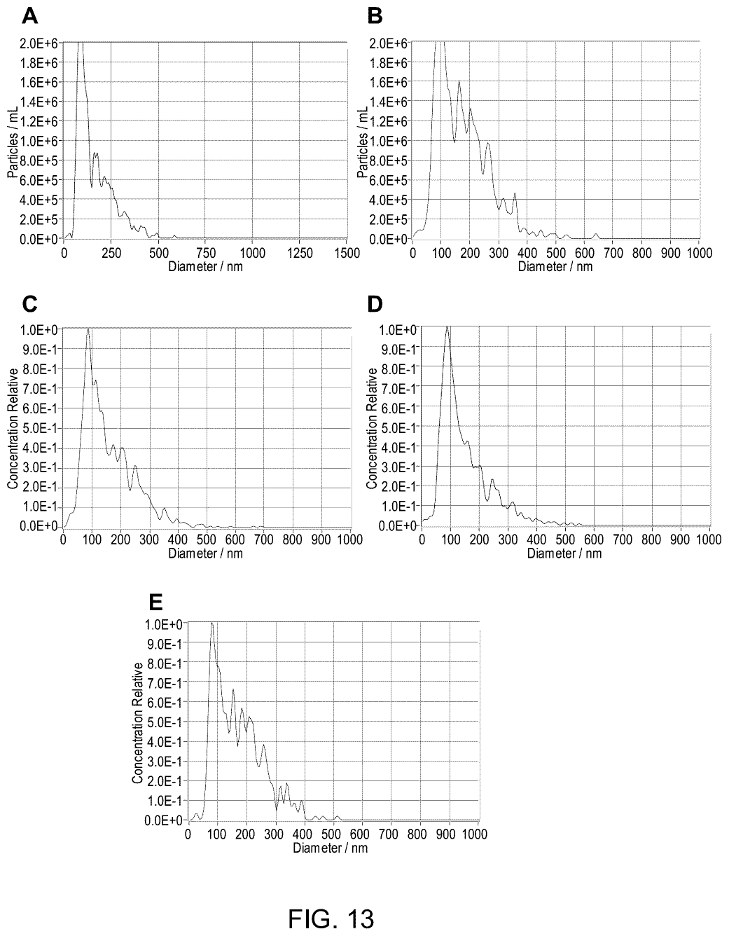

A spectrophotometric method was used to validate the presence of peptidoglycan hydrolase as a potential mechanism for antimicrobial activity and EVs entering the cell. The results showed that peptidoglycan was degraded by peptidoglycan hydrolase of fraction 3 (F3) EVs from selected strains (FIG. 12). Kinetics showed that hydrolase can react with peptidoglycan after 30 minutes of incubation in EVs while lysozyme start the reaction after 20 minutes.

### EVs Demonstrate a Strain Specific Dose-Dependent Antimicrobial Effect

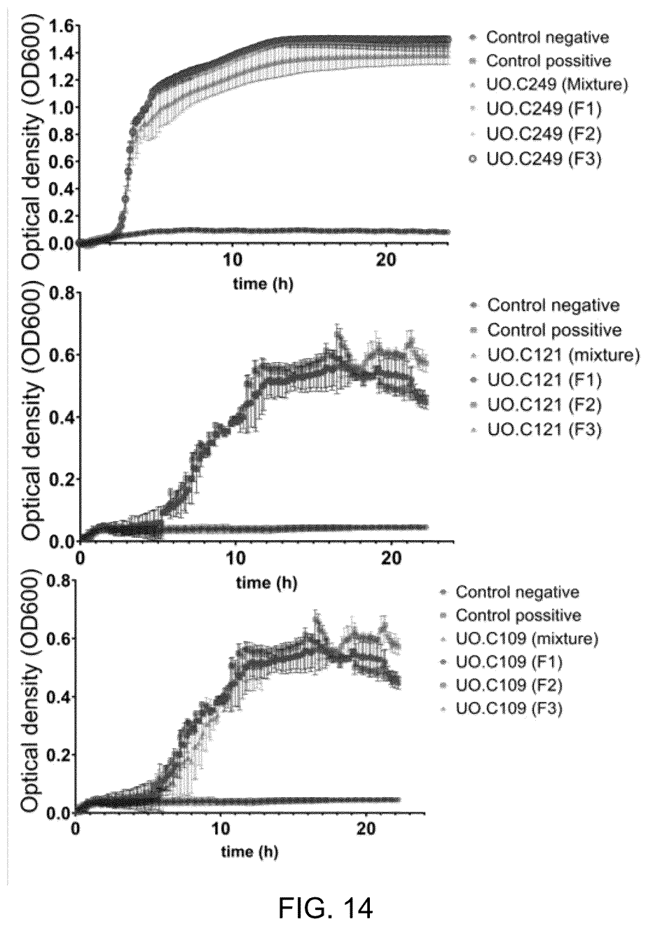

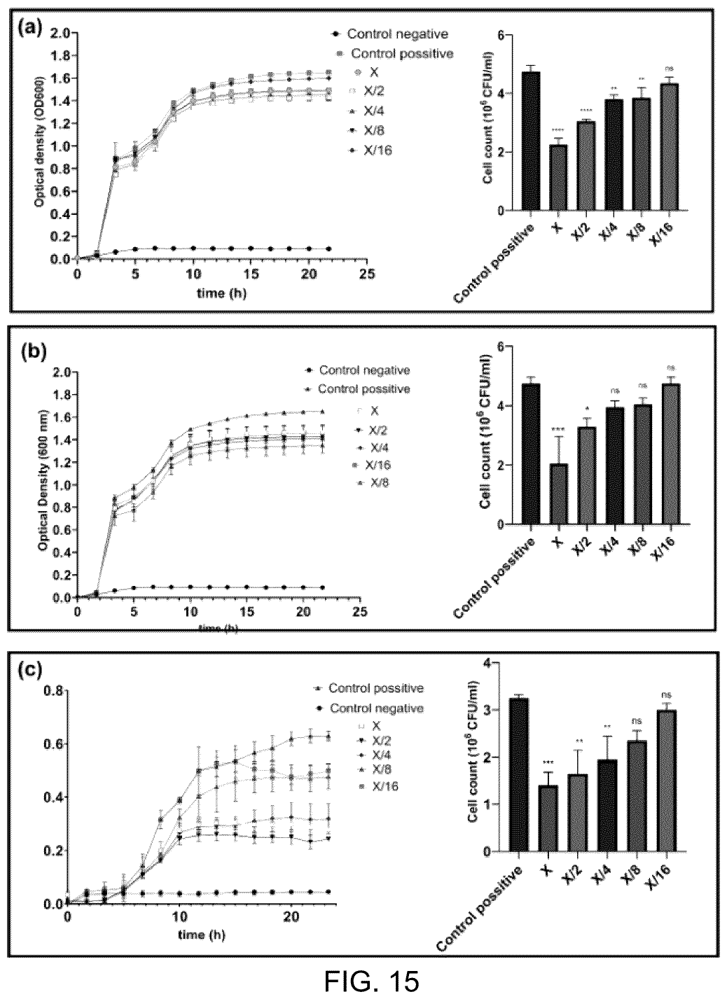

To assess the impact of the EV dose on the inhibition of selected pathogens, EV samples (1X=1.0E+10 particles per mL) were diluted at different concentrations (X, X/2, X/4, X/8, and X/16). The preliminary results confirmed that the CFS and EVs from Lg. salivarius UO.C109, and Lg. saerimneri UO.C121 inhibited S. typhimurium but not C. jejuni. In contrast, EVs from Lg. salivarius UO.C249 exhibited antibacterial activity only against C. jejuni. These results indicate that EVs from Lg. salivarius UO.C249 was the most potent against C. jejuni (FIG. 15C). Indeed, bacterial growth inhibition was approximately 10-20% at various EV concentrations for Lg. salivarius UO.C109, and Lg. saerimneri UO.C121 against S. typhimurium (FIG. 15AB), whereas EV produced by Lg. salivarius UO.C249 induced up to 70% inhibition of C. jejuni (FIG. 15C). As illustrated in FIG. 15, EVs diluted at X/16 did not exhibit significant antimicrobial activity compared with the positive control (S. typhimurium culture without any treatment) (P=0.37). Colony counting confirmed a dose-response effect of the tested EVs on their target pathogens (FIG. 15).

### EV Subpopulations Downregulated the Expression of Virulence Factors in Targeted Pathogens

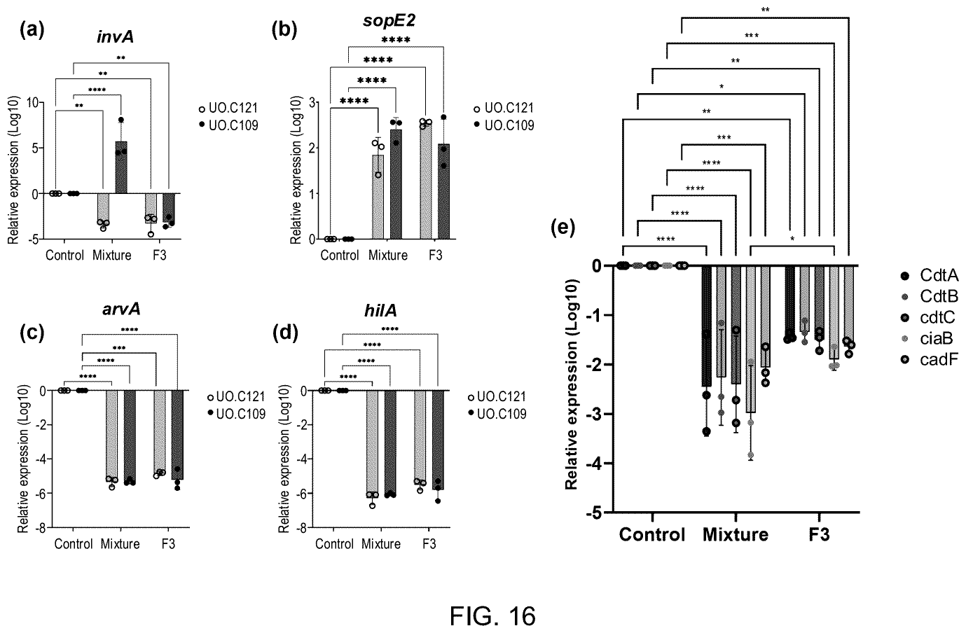

The expression of genes known to be virulence factors in S. typhimurium and C. jejuni was tested after exposure to the EVs mixture or fraction F3, as depicted in FIG. 16. These factors include the type III Secretion System (T3SS), adhesion molecules, invasion proteins, and resistance to host defenses, all of which contribute to their ability to cause disease. Our results in FIG. 6 show the downregulation of most genes in both pathogens (avrA, invA, and hilA in S. typhimurium and CdtA, CdtB, CdtC, ciaB, and cadF in C. jejuni) upon exposure to the EVs mixture or F3 fraction. Although sopE2 expression was upregulated in samples treated with the EVs mixture (1.8-fold for samples treated with EVs from Lg. saerimneri UO.C121 and 2.4-fold for EVs from Lg. salivarius UO.C109), or F3 (2.5-fold for the sample treated with EVs from Lg. saerimneri UO.C121 and 2.8-fold for EVs from Lg. salivarius UO.C109). In addition, inva in the sample treated with a mixture of EVs was upregulated (5.7-fold for EVs from Lg. salivarius UO.C109) compared to the control sample; however, samples treated with F3 showed a decrease in expression level by 3.1-fold.

### REFERENCES

Abd El-Hack, M. E., Samak, D. H., Noreldin, A. E., El-Naggar, K., and Abdo, M. (2018). Probiotics and plant-derived compounds as eco-friendly agents to inhibit microbial toxins in poultry feed: a comprehensive review. Environ. Sci. Pollut. Res. 25, 31971-31986.
Abukhattab, S., Taweel, H., Awad, A., Crump, L., Vonaesch, P., Zinsstag, J., et al. (2022). Systematic Review and Meta-Analysis of Integrated Studies on Salmonella and Campylobacter Prevalence, Serovar, and Phenotyping and Genetic of Antimicrobial Resistance in the Middle East-A One Health Perspective. Antibiotics 11, 536.
Adhikari, B., and Kwon, Y. M. (2017). Characterization of the culturable subpopulations of Lactobacillus in the chicken intestinal tract as a resource for probiotic development. Front. Microbiol. 8, 1389.
Albuquerque, P. C., Nakayasu, E. S., Rodrigues, M. L., Frases, S., Casadevall, A., Zancope?Oliveira, R. M., et al. (2008). Vesicular transport in Histoplasma capsulatum: an effective mechanism for trans?cell wall transfer of proteins and lipids in ascomycetes. Cellular microbiology 10, 1695-1710.
Barrett, E., Ross, R., O'Toole, P. W., Fitzgerald, G. F., and Stanton, C. (2012). ??Aminobutyric acid production by culturable bacteria from the human intestine. J. Appl. Microbiol. 113, 411-417.
Benítez-Chao, D. F., Leon-Buitimea, A., Lerma-Escalera, J. A., and Morones-Ramírez, J. R. (2021). Bacteriocins: an overview of antimicrobial, toxicity, and biosafety assessment by in vivo models. Front. Microbiol. 12, 630695.
Berndt, A., Wilhelm, A., Jugert, C., Pieper, J., Sachse, K., and Methner, U. (2007). Chicken cecum immune response to Salmonella enterica serovars of different levels of invasiveness. Infect. Immun. 75, 5993-6007.
Bolyen, E., Rideout, J. R., Dillon, M. R., Bokulich, N. A., Abnet, C. C., AI-Ghalith, G. A., et al. (2019). Reproducible, interactive, scalable and extensible microbiome data science using QIIME 2. Nat. Biotechnol. 37, 852-857. doi: 10.1038/s41587-019-0209-9.
Briaud, P., and Carroll, R. K. (2020). Extracellular vesicle biogenesis and functions in gram-positive bacteria. Infection and immunity 88, 10-1128.
Brown, L., Wolf, J. M., Prados-Rosales, R., and Casadevall, A. (2015). Through the wall: extracellular vesicles in Gram-positive bacteria, mycobacteria and fungi. Nature Reviews Microbiology 13, 620-630.
Buist, G., Steen, A., Kok, J., and Kuipers, O. P. (2008). LysM, a widely distributed protein motif for binding to (peptido) glycans. Molecular microbiology 68, 838-847.
Bullard, J., St-Gelais, D., and Turgeon, S. L. (2018). Production of set yoghurts using thermophilic starters composed of two strains with different growth biocompatibilities and producing different exopolysaccharides. Int. Dairy J. 79, 33-42.
Burkholder, K. M., and Bhunia, A. K. (2009). Salmonella enterica serovar Typhimurium adhesion and cytotoxicity during epithelial cell stress is reduced by Lactobacillus rhamnosus GG. Gut Pathog. 1, 1-10.
Callahan, B. J., McMurdie, P. J., Rosen, M. J., Han, A. W., Johnson, A. J. A., and Holmes, S. P. (2016). DADA2: High-resolution sample inference from Illumina amplicon data. Nat. Methods 13, 581-583. doi: 10.1038/nmeth.3869.
Campedelli, I., Mathur, H., Salvetti, E., Clarke, S., Rea, M. C., Torriani, S., et al. (2018). Genus-Wide Assessment of Antibiotic Resistance in Lactobacillus spp. Appl. Environ. Microbiol. 85, e01738-18, /aem/85/1/AEM.01738-18.atom. doi: 10.1128/AEM.01738-18.
Cao, Y. (2023). microbiomeMarker: microbiome biomarker analysis toolkit. doi: 10.18129/B9.bioc.microbiomeMarker.
Caruana, J. C., and Walper, S. A. (2020). Bacterial membrane vesicles as mediators of microbe-microbe and microbe-host community interactions. Frontiers in microbiology 11, 432.
Cavicchioli, V., Camargo, A., Todorov, S., and Nero, L. (2017). Novel bacteriocinogenic Enterococcus hirae and Pediococcus pentosaceus strains with antilisterial activity isolated from Brazilian artisanal cheese. J. Dairy Sci. 100, 2526-2535.
Chai, S., Cole, D., Nisler, A., and Mahon, B. E. (2017). Poultry: The most common food in outbreaks with known pathogens, United States, 1998-2012. Epidemiol. Infect. 145, 316-325.
Chatterjee, D., and Chaudhuri, K. (2011). Association of cholera toxin with Vibrio cholerae outer membrane vesicles which are internalized by human intestinal epithelial cells. FEBS letters 585, 1357-1362.
Chatterjee, S., and Das, J. (1967). Electron microscopic observations on the excretion of cell-wall material by Vibrio cholerae. Microbiology 49, 1-11.
Chichlowski, M., Croom, W. J., Edens, F. W., McBride, B. W., Qiu, R., Chiang, C. C., et al. (2007). Microarchitecture and spatial relationship between bacteria and ileal, cecal, and colonic epithelium in chicks fed a direct-fed microbial, PrimaLac, and salinomycin. Poult. Sci. 86, 1121-1132. doi: 10.1093/ps/86.6.1121.
Chlebicz, A., and Śliżewska, K. (2020). In vitro detoxification of aflatoxin B 1, deoxynivalenol, fumonisins, T-2 toxin and zearalenone by probiotic bacteria from genus Lactobacillus and Saccharomyces cerevisiae yeast. Probiotics and antimicrobial proteins 12, 289-301.
Cox, J., and Mann, M. (2008). MaxQuant enables high peptide identification rates, individualized ppb-range mass accuracies and proteome-wide protein quantification. Nat. Biotechnol. 26, 1367-1372.
Cox, J., and Mann, M. (2008). MaxQuant enables high peptide identification rates, individualized ppb-range mass accuracies and proteome-wide protein quantification. Nature biotechnology 26, 1367-1372.
Cox, J., Hein, M. Y., Luber, C. A., Paron, I., Nagaraj, N., and Mann, M. (2014). Accurate proteome-wide label-free quantification by delayed normalization and maximal peptide ratio extraction, termed MaxLFQ. Molecular & cellular proteomics 13, 2513-2526.
Cox, J., Neuhauser, N., Michalski, A., Scheltema, R. A., Olsen, J. V., and Mann, M. (2011). Andromeda: a peptide search engine integrated into the MaxQuant environment. Journal of proteome research 10, 1794-1805.
Croatti, V., Parolin, C., Giordani, B., Foschi, C., Fedi, S., and Vitali, B. (2022). Lactobacilli extracellular vesicles: potential postbiotics to support the vaginal microbiota homeostasis. Microbial Cell Factories 21, 1-11.
da Silva Sabo, S., Vitolo, M., González, J. M. D., and de Souza Oliveira, R. P. (2014). Overview of Lactobacillus plantarum as a promising bacteriocin producer among lactic acid bacteria. Food Res. Int. 64, 527-536.
da Silva, D. R., Gonzalez, C. F., and Lorca, G. (2023). Internalization of extracellular vesicles from Lactobacillus johnsonii N6. 2 elicit an RNA sensory response in human pancreatic cell lines. Journal of Extracellular Biology 2, e101.
Dean, S. N., Rimmer, M. A., Turner, K. B., Phillips, D. A., Caruana, J. C., Hervey I V, W. J., et al. (2020). Lactobacillus acidophilus membrane vesicles as a vehicle of bacteriocin delivery. Frontiers in Microbiology 11, 710.
Delcher, A. L., Harmon, D., Kasif, S., White, O., and Salzberg, S. L. (1999). Improved microbial gene identification with GLIMMER. Nucleic Acids Res. 27, 4636-4641.
Dziarski, R., and Gupta, D. (2006). The peptidoglycan recognition proteins (PGRPs). Genome biology 7, 232.
Eckburg, P. B., Bik, E. M., Bernstein, C. N., Purdom, E., Dethlefsen, L., Sargent, M., et al. (2005). Diversity of the human intestinal microbial flora. science 308, 1635-1638.
EFSA Panel on Additives and Products or Substances used in Animal Feed (FEEDAP) (2012). Guidance on the assessment of bacterial susceptibility to antimicrobials of human and veterinary importance. EFSA J. 10, 2740.
Fey, A., Eichler, S., Flavier, S., Christen, R., Höfle, M. G., and Guzmán, C. A. (2004). Establishment of a real-time PCR-based approach for accurate quantification of bacterial RNA targets in water, using Salmonella as a model organism. Appl. Environ. Microbiol. 70, 3618-3623.
Garcia-Cano, I., Rocha-Mendoza, D., Ortega-Anaya, J., Wang, K., Kosmerl, E., and Jiménez-Flores, R. (2019). Lactic acid bacteria isolated from dairy products as potential producers of lipolytic, proteolytic and antibacterial proteins. Appl. Microbiol. Biotechnol. 103, 5243-5257.
Gardner-Fortier, C., St-Gelais, D., Champagne, C. P., and Vuillemard, J.-C. (2013). Determination of optimal conditions for ?-aminobutyric acid production by Lactococcus lactis ssp. lactis. Int. Dairy J. 32, 136-143.
Guo, S., Xi, Y., Xia, Y., Wu, T., Zhao, D., Zhang, Z., et al. (2021). Dietary Lactobacillus fermentum and Bacillus coagulans supplementation modulates intestinal immunity and microbiota of broiler chickens challenged by Clostridium perfringens. Front. Vet. Sci. 8, 680742.
Hammami, R., Fernandez, B., Lacroix, C., and Fliss, I. (2013). Anti-infective properties of bacteriocins: an update. Cell. Mol. Life Sci. 70, 2947-2967. doi: 10.1007/s00018-012-1202-3.
Hanchi, H., Hammami, R., Fernandez, B., Kourda, R., Ben Hamida, J., and Fliss, I. (2016). Simultaneous production of formylated and nonformylated enterocins L50A and L50B as well as 61A, a new glycosylated durancin, by Enterococcus durans 61A, a strain isolated from artisanal fermented milk in Tunisia. J. Agric. Food Chem. 64, 3584-3590.
Hanchi, H., Mottawea, W., Sebei, K., and Hammami, R. (2018). The genus Enterococcus: between probiotic potential and safety concerns—an update. Front. Microbiol. 9, 1791.
Helmy, Y. A., Taha-Abdelaziz, K., Hawwas, H. A. E.-H., Ghosh, S., AlKafaas, S. S., Moawad, M. M., et al. (2023). Antimicrobial Resistance and Recent Alternatives to Antibiotics for the Control of Bacterial Pathogens with an Emphasis on Foodborne Pathogens. Antibiotics 12, 274.
Hernández-González, J. C., Martinez-Tapia, A., Lazcano-Hernández, G., Garcïa-Perez, B. E., and Castrejón-Jiménez, N. S. (2021). Bacteriocins from lactic acid bacteria. A powerful alternative as antimicrobials, probiotics, and immunomodulators in veterinary medicine. Animals 11, 979.
Hofacre, C. L., Smith, J. A., and Mathis, G. F. (2018). An optimist's view on limiting necrotic enteritis and maintaining broiler gut health and performance in today's marketing, food safety, and regulatory climate. Poult. Sci. 97, 1929-1933.
Hu, J., Ma, L., Nie, Y., Chen, J., Zheng, W., Wang, X., et al. (2018). A Microbiota-Derived Bacteriocin Targets the Host to Confer Diarrhea Resistance in Early-Weaned Piglets. Cell Host Microbe 24, 817-832.e8. doi: 10.1016/j.chom.2018.11.006.
Kempf, F., Menanteau, P., Rychlik, I., Kubasová, T., Trotereau, J., Virlogeux?Payant, I., et al. (2020). Gut microbiota composition before infection determines the Salmonella super?and low?shedder phenotypes in chicken. Microb. Biotechnol. 13, 1611-1630.
Kers, J. G., Velkers, F. C., Fischer, E. A., Hermes, G. D., Stegeman, J. A., and Smidt, H. (2018). Host and environmental factors affecting the intestinal microbiota in chickens. Front. Microbiol. 9, 235.
Khan, S., and Chousalkar, K. K. (2021). Functional enrichment of gut microbiome by early supplementation of Bacillus based probiotic in cage free hens: a field study. Anim. Microbiome 3, 1-18.
Khan, S., Moore, R. J., Stanley, D., and Chousalkar, K. K. (2020). The gut microbiota of laying hens and its manipulation with prebiotics and probiotics to enhance gut health and food safety. Appl. Environ. Microbiol. 86, e00600-20.
Koninkx, J. F., Tooten, P. C., and Malago, J. J. (2010). Probiotic bacteria induced improvement of the mucosal integrity of enterocyte-like Caco-2 cells after exposure to Salmonella enteritidis 857. J. Funct. Foods 2, 225-234.
Kruger, M. F., de Souza Barbosa, M., Miranda, A., Landgraf, M., Destro, M. T., Todorov, S. D., et al. (2013). Isolation of bacteriocinogenic strain of Lactococcus lactis subsp. lactis from Rocket salad (Eruca sativa Mill.) and evidences of production of a variant of nisin with modification in the leader-peptide. Food Control 33, 467-476.
Lee, B.-H., Chen, Y.-Z., Shen, T.-L., Pan, T.-M., and Hsu, W.-H. (2023). Proteomic characterization of extracellular vesicles derived from lactic acid bacteria. Food Chemistry, 136685.
Lee, E., Choi, D., Kim, D., Kim, J., Park, J. O., Kim, S., et al. (2009). Gram?positive bacteria produce membrane vesicles: proteomics?based characterization of Staphylococcus aureus?derived membrane vesicles. Proteomics 9, 5425-5436.
Leyva-Diaz, A. A., Hernandez-Patlan, D., Solis-Cruz, B., Adhikari, B., Kwon, Y. M., Latorre, J. D., et al. (2021). Evaluation of curcumin and copper acetate against Salmonella typhimurium infection, intestinal permeability, and cecal microbiota composition in broiler chickens. J. Anim. Sci. Biotechnol. 12, 1-12.
Lin, C.-K., Tsai, H.-C., Lin, P.-P., Tsen, H.-Y., and Tsai, C.-C. (2008). Lactobacillus acidophilus LAP5 able to inhibit the Salmonella choleraesuis invasion to the human Caco-2 epithelial cell. Anaerobe 14, 251-255.
Liu, L., Li, Q., Yang, Y., and Guo, A. (2021). Biological function of short-chain fatty acids and its regulation on intestinal health of poultry. Front. Vet. Sci. 8, 736739.
Lone, A., Mottawea, W., Ait Chait, Y., and Hammami, R. (2021). Dual inhibition of Salmonella enterica and Clostridium perfringens by new probiotic candidates isolated from chicken intestinal mucosa. Microorganisms 9, 166.
Lone, A., Mottawea, W., Mehdi, Y., and Hammami, R. (2022). Bacteriocinogenic probiotics as an integrated alternative to antibiotics in chicken production—why and how? Crit. Rev. Food Sci. Nutr. 62, 8744-8760. doi: 10.1080/10408398.2021.1932722.
Manyi-Loh, C., Mamphweli, S., Meyer, E., and Okoh, A. (2018). Antibiotic use in agriculture and its consequential resistance in environmental sources: potential public health implications. Molecules 23, 795.
Markowiak-Kope?, P., and ?li?ewska, K. (2020). The effect of probiotics on the production of short-chain fatty acids by human intestinal microbiome. Nutrients 12, 1107.
Martin-Visscher, L. A., Gong, X., Duszyk, M., and Vederas, J. C. (2009). The three-dimensional structure of carnocyclin A reveals that many circular bacteriocins share a common structural motif. Journal of Biological Chemistry 284, 28674-28681.
McMillan, H. M., and Kuehn, M. J. (2022). Proteomic Profiling Reveals Distinct Bacterial Extracellular Vesicle Subpopulations with Possibly Unique Functionality. Applied and Environmental Microbiology, e01686-22.
McMurdie, P. J., and Holmes, S. (2013). phyloseq: An R Package for Reproducible Interactive Analysis and Graphics of Microbiome Census Data. PLoS ONE 8, e61217. doi: 10.1371/journal.pone.0061217.
Messaoudi, S., Manai, M., Kergourlay, G., Prevost, H., Connil, N., Chobert, J.-M., et al. (2013). Lactobacillus salivarius: bacteriocin and probiotic activity. Food Microbiol. 36, 296-304.
Miglani, R., Parveen, N., Kumar, A., Dewali, S., Rawat, G., Mishra, R., et al. (2023). “Bacteriocin and its biomedical application with special reference to Lactobacillus,” in Recent Advances and Future Perspectives of Microbial Metabolites (Elsevier), 123-146.
Minic, Z., Li, Y., Huttmann, N., Uppal, G. K., D'Mello, R., and Berezovski, M. V. (2023). Lysine Acetylome of Breast Cancer-Derived Small Extracellular Vesicles Reveals Specific Acetylation Patterns for Metabolic Enzymes. Biomedicines 11, 1076.
Mojgani, N., Sabiri, G., Ashtiani, M., and Torshizi, M. (2009). Characterization of bacteriocins produced by Lactobacillus brevis NM 24 and L. fermentum NM 332 isolated from green olives in Iran. Int J Microbiol 6.
Mon, K. K., Saelao, P., Halstead, M. M., Chanthavixay, G., Chang, H.-C., Garas, L., et al. (2015). Salmonella enterica serovars Enteritidis infection alters the indigenous microbiota diversity in young layer chicks. Front. Vet. Sci. 2, 61.
Mon, K. K., Zhu, Y., Chanthavixay, G., Kern, C., and Zhou, H. (2020). Integrative analysis of gut microbiome and metabolites revealed novel mechanisms of intestinal Salmonella carriage in chicken. Sci. Rep. 10, 1-14.
Mousavi, R., Mottawea, W., Audet, M.-C., and Hammami, R. (2022). Survival and Interplay of ?-Aminobutyric Acid-Producing Psychobiotic Candidates with the Gut Microbiota in a Continuous Model of the Human Colon. Biology 11, 1311.
Nagaraj, N., Kulak, N. A., Cox, J., Neuhauser, N., Mayr, K., Hoerning, O., et al. (2012). System-wide perturbation analysis with nearly complete coverage of the yeast proteome by single-shot ultra HPLC runs on a bench top Orbitrap. Molecular & Cellular Proteomics 11.
Nakayama, M., Shimatani, K., Ozawa, T., Shigemune, N., Tomiyama, D., Yui, K., et al. (2015). Mechanism for the antibacterial action of epigallocatechin gallate (EGCg) on Bacillus subtilis. Bioscience, Biotechnology, and Biochemistry 79, 845-854.
O'Callaghan, J., Butto, L. F., MacSharry, J., Nally, K., and O'Toole, P. W. (2012). Influence of adhesion and bacteriocin production by Lactobacillus salivarius on the intestinal epithelial cell transcriptional response. Appl. Environ. Microbiol. 78, 5196-5203.
Oh J K, Vasquez R, Kim S H, Hwang I C, Song J H, Park J H, Kim I H, Kang D K. Multispecies probiotics alter fecal short-chain fatty acids and lactate levels in weaned pigs by modulating gut microbiota. J Anim Sci Technol. 2021 September; 63(5):1142-1158. doi: 10.5187/jast.2021.e94. Epub 2021 Sep. 30. PMID: 34796353; PMCID: PMC8564300
Oksanen, J., Simpson, G. L., Blanchet, F. G., Kindt, R., Legendre, P., Minchin, P. R., et al. (2022). vegan: Community Ecology Package. Available at: https://CRAN.R-project.org/package=vegan [Accessed Apr. 6, 2023].
Onódi, Z., Pelyhe, C., Terezia Nagy, C., Brenner, G. B., Almdsi, L., Kittel, A., et al. (2018). Isolation of high-purity extracellular vesicles by the combination of iodixanol density gradient ultracentrifugation and bind-elute chromatography from blood plasma. Frontiers in physiology 9, 1479.
O'Shea, E. F., O'Connor, P. M., Raftis, E. J., O'Toole, P. W., Stanton, C., Cotter, P. D., et al. (2011). Production of multiple bacteriocins from a single locus by gastrointestinal strains of Lactobacillus salivarius. J. Bacteriol. 193, 6973-6982.
Pedroso, A. A., Lee, M. D., and Maurer, J. J. (2021). Strength lies in diversity: how community diversity limits salmonella abundance in the chicken intestine. Front. Microbiol. 12, 694215.
Pingitore, E. V., Hébert, E. M., Nader-Maclas, M. E., and Sesma, F. (2009). Characterization of salivaricin CRL 1328, a two-peptide bacteriocin produced by Lactobacillus salivarius CRL 1328 isolated from the human vagina. Res. Microbiol. 160, 401-408.
Prado-Acosta, M., Ruzal, S. M., Allievi, M. C., Palomino, M. M., and Sanchez Rivas, C. (2010). Synergistic effects of the Lactobacillus acidophilus surface layer and nisin on bacterial growth. Appl. Environ. Microbiol. 76, 974-977.
Quast, C., Pruesse, E., Yilmaz, P., Gerken, J., Schweer, T., Yarza, P., et al. (2013). The SILVA ribosomal RNA gene database project: improved data processing and web-based tools. Nucleic Acids Res. 41, D590-596. doi: 10.1093/nar/gks1219.
Risha, Y., Minic, Z., Ghobadloo, S. M., and Berezovski, M. V. (2020). The proteomic analysis of breast cell line exosomes reveals disease patterns and potential biomarkers. Sci. Rep. 10, 13572.
Robinson, K., Assumpcao, A. L., Arsi, K., Erf, G. F., Donoghue, A., and Jesudhasan, P. R. (2022). Effect of Salmonella typhimurium Colonization on Microbiota Maturation and Blood Leukocyte Populations in Broiler Chickens. Animals 12, 2867.
Rodrigues, M. L., Nakayasu, E. S., Oliveira, D. L., Nimrichter, L., Nosanchuk, J. D., Almeida, I. C., et al. (2008). Extracellular vesicles produced by Cryptococcus neoformans contain protein components associated with virulence. Eukaryotic cell 7, 58-67.
Schaack, B., Hindré, T., Quansah, N., Hannani, D., Mercier, C., and Laurin, D. (2022). Microbiota-Derived Extracellular Vesicles Detected in Human Blood from Healthy Donors. IJMS 23, 13787. doi: 10.3390/ijms232213787.
Segata, N., Izard, J., Waldron, L., Gevers, D., Miropolsky, L., Garrett, W. S., et al. (2011). Metagenomic biomarker discovery and explanation. Genome Biol. 12, R60. doi: 10.1186/gb-2011-12-6-r60.
Sidhom, K., Obi, P. O., and Saleem, A. (2020). A review of exosomal isolation methods: is size exclusion chromatography the best option? International Journal of Molecular Sciences 21, 6466.
Song, J., Li, Q., Everaert, N., Liu, R., Zheng, M., Zhao, G., et al. (2020). Dietary inulin supplementation modulates short-chain fatty acid levels and cecum microbiota composition and function in chickens infected with salmonella. Front. Microbiol. 11, 584380.
Soult, M. C., Lonergan, N. E., Shah, B., Kim, W.-K., Britt, L., and Sullivan, C. J. (2013). Outer membrane vesicles from pathogenic bacteria initiate an inflammatory response in human endothelial cells. journal of surgical research 184, 458-466.
Stern, N., Svetoch, E., Eruslanov, B., Perelygin, V., Mitsevich, E., Mitsevich, I., et al. (2006). Isolation of a Lactobacillus salivarius strain and purification of its bacteriocin, which is inhibitory to Campylobacter jejuni in the chicken gastrointestinal system. Antimicrob. Agents Chemother. 50, 3111-3116.
Svetoch, E. A., Eruslanov, B. V., Levchuk, V. P., Perelygin, V. V., Mitsevich, E. V., Mitsevich, I. P., et al. (2011). Isolation of Lactobacillus salivarius 1077 (NRRL B-50053) and characterization of its bacteriocin, including the antimicrobial activity spectrum. Appl. Environ. Microbiol. 77, 2749-2754.
Tam, R., and Saier Jr, M. H. (1993). Structural, functional, and evolutionary relationships among extracellular solute-binding receptors of bacteria. Microbiological reviews 57, 320-346.
Tang, Y., Underwood, A., Gielbert, A., Woodward, M. J., and Petrovska, L. (2014). Metaproteomics analysis reveals the adaptation process for the chicken gut microbiota. Appl. Environ. Microbiol. 80, 478-485.
Trikha, R., Rishi, P., and Tewari, R. (2017). Remediation of intramacrophageal Shigella dysenteriae type 1 by probiotic lactobacilli isolated from human infants' stool samples. Indian J. Med. Res. 145, 679.
Valencia, S. (2019). OmicsBox-Bioinformatics made easy. Mar. 3, 2019.
Vallejo, M. C., Nakayasu, E. S., Longo, L. V., Ganiko, L., Lopes, F. G., Matsuo, A. L., et al. (2012). Lipidomic analysis of extracellular vesicles from the pathogenic phase of Paracoccidioides brasiliensis. PloS one 7, e39463.
Van Deun, J., Mestdagh, P., Sormunen, R., Cocquyt, V., Vermaelen, K., Vandesompele, J., et al. (2014). The impact of disparate isolation methods for extracellular vesicles on downstream RNA profiling. Journal of extracellular vesicles 3, 24858.
Van Niel, G., d'Angelo, G., and Raposo, G. (2018). Shedding light on the cell biology of extracellular vesicles. Nature reviews Molecular cell biology 19, 213-228.
Villalba Primitz, J., Vázquez, S., Ruberto, L., Lo Balbo, A., and Mac Cormack, W. (2021). Bioremediation of hydrocarbon-contaminated soil from Carlini Station, Antarctica: effectiveness of different nutrient sources as biostimulation agents. Polar Biology 44, 289-303.
Vimont, A., Fernandez, B., Hammami, R., Ababsa, A., Daba, H., and Fliss, I. (2017). Bacteriocin-Producing Enterococcus faecium LCW 44: A High Potential Probiotic Candidate from Raw Camel Milk. Front. Microbiol. 8, 865. doi: 10.3389/fmicb.2017.00865.
Wang, L., Li, L., Lv, Y., Chen, Q., Feng, J., and Zhao, X. (2018). Lactobacillus plantarum restores intestinal permeability disrupted by Salmonella infection in newly-hatched chicks. Sci. Rep. 8, 1-10.
Wang, Q., Gong, J., Huang, X., Yu, H., and Xue, F. (2009). In vitro evaluation of the activity of microencapsulated carvacrol against Escherichia coli with K88 pili. J. Appl. Microbiol. 107, 1781-1788.
White, D. G., Zhao, S., Simjee, S., Wagner, D. D., and McDermott, P. F. (2002). Antimicrobial resistance of foodborne pathogens. Microbes and infection 4, 405-412.
Wi?niewski, J. R., Zougman, A., Nagaraj, N., and Mann, M. (2009). Universal sample preparation method for proteome analysis. Nature methods 6, 359-362.
Wickham, H. (2016). ggplot2: Elegant Graphics for Data Analysis. Springer-Verlag New York Available at: https://ggplot2.tidyverse.org.
Xiong, W., Wang, Y., Sun, Y., Ma, L., Zeng, Q., Jiang, X., et al. (2018). Antibiotic-mediated changes in the fecal microbiome of broiler chickens define the incidence of antibiotic resistance genes. Microbiome 6, 1-11.
Xu, Y., Li, G., Zhang, B., Wu, Q., Wang, X., and Xia, X. (2015). Tannin-rich pomegranate rind extracts reduce adhesion to and invasion of Caco-2 cells by Listeria monocytogenes and decrease its expression of virulence genes. J. Food Prot. 78, 128-133.
Yamazaki, M., Ohtsu, H., Yakabe, Y., Kishima, M., and Abe, H. (2012). In vitro screening of lactobacilli isolated from chicken excreta to control Salmonella Enteritidis and Typhimurium. Br. Poult. Sci. 53, 183-189.
Yang, S., Wen, X., Zhao, L., Shi, Y., and Jin, H. (2014). Crude oil treatment leads to shift of bacterial communities in soils from the deep active layer and upper permafrost along the China-Russia crude oil pipeline route. PloS One 9, e96552.
Yang, Y., Liu, Y., Zhou, S., Huang, L., Chen, Y., and Huan, H. (2019). Bile salt hydrolase can improve Lactobacillus plantarum survival in gastrointestinal tract by enhancing their adhesion ability. FEMS Microbiol. Lett. 366, fnz100.
Yilmaz, P., Parfrey, L. W., Yarza, P., Gerken, J., Pruesse, E., Quast, C., et al. (2014). The SILVA and “All-species Living Tree Project (LTP)” taxonomic frameworks. Nucleic Acids Res. 42, D643-D648. doi: 10.1093/nar/gkt1209.
Yu, H., Ding, X., Li, N., Zhang, X., Zeng, X., Wang, S., et al. (2017). Dietary supplemented antimicrobial peptide microcin J25 improves the growth performance, apparent total tract digestibility, fecal microbiota, and intestinal barrier function of weaned pigs. J. Anim. Sci. 95, 5064-5076.
Zhang, B., Li, G., Shahid, M. S., Gan, L., Fan, H., Lv, Z., et al. (2020). Dietary I-arginine supplementation ameliorates inflammatory response and alters gut microbiota composition in broiler chickens infected with Salmonella enterica serovar Typhimurium. Poult. Sci. 99, 1862-1874.
Kumar, M., Dhaka, P., Vijay, D., Vergis, J., Mohan, V., Kumar, A., . . . & Rawool, D. B. (2016). Antimicrobial effects of Lactobacillus plantarum and Lactobacillus acidophilus against multidrug-resistant enteroaggregative Escherichia coli. International journal of antimicrobial agents, 48(3), 265-270.
Hu, C. H., Ren, L. Q., Zhou, Y., & Ye, B. C. (2019). Characterization of antimicrobial activity of three Lactobacillus plantarum strains isolated from Chinese traditional dairy food. Food science & nutrition, 7(6), 1997-2005.
Zhang, J., Gao, J., Guo, Y., Wu, Z., & Pan, D. (2017). Extraction of Lactobacillus acidophilus CICC 6074 S-layer proteins and their ability to inhibit enteropathogenic Escherichia coli. Current microbiology, 74, 1123-1129.
De LeBlanc, A. D. M., Castillo, N. A., & Perdigon, G. (2010). Anti-infective mechanisms induced by a probiotic Lactobacillus strain against Salmonella enterica serovar Typhimurium infection. International Journal of Food Microbiology, 138(3), 223-231.
Sun, L., Zhao, H., Liu, L., Wu, X., Gao, Q., & Zhao, Y. (2018). Effects of Lactobacillus on the inhibition of Helicobacter pylori growth. Biotechnology & Biotechnological Equipment, 32(6), 1533-1540.
Mukherjee, S., & Ramesh, A. (2015). Bacteriocin-producing strains of Lactobacillus plantarum inhibit adhesion of Staphylococcus aureus to extracellular matrix: quantitative insight and implications in antibacterial therapy. Journal of Medical Microbiology, 64(12), 1514-1526.
Ocaña, V. S., Pesce de Ruiz Holgado, A. A., & Nader-Macias, M. E. (1999). Characterization of a bacterocin-like substance produced by a vaginal Lactobacillus salivarius strain. Applied and environmental microbiology, 65(12), 5631-5635.
Liévin-Le Moal, V., & Servin, A. L. (2014). Anti-infective activities of Lactobacillus strains in the human intestinal microbiota: from probiotics to gastrointestinal anti-infectious biotherapeutic agents. Clinical microbiology reviews, 27(2), 167-199.
Song, M., Yun, B., Moon, J. H., Park, D. J., Lim, K., & Oh, S. (2015). Characterization of selected Lactobacillus strains for use as probiotics. Korean journal for food science of animal resources, 35(4), 551.
Llamas, M., Greses, S., Magdalena, J. A., González-Fernández, C., & Tomás-Pejó, E. (2023). Microbial co-cultures for biochemicals production from lignocellulosic biomass: A review. Bioresource Technology, 386, 129499.
Prabhu, G., Bhat, D., Bhat, R. M., & Selvaraj, S. (2022). A critical look at bioproducts co-cultured under solid state fermentation and their challenges and industrial applications. Waste and Biomass Valorization, 13(7), 3095-3111.
Hatti-Kaul, R., Chen, L., Dishisha, T., & Enshasy, H. E. (2018). Lactic acid bacteria: From starter cultures to producers of chemicals. FEMS Microbiology Letters, 365(20), fny213.
Santo, L. D. É., Ishii, M., Pinto, U. M., Matsudo, M. C., & Carvalho, J. C. M. D. (2022). Obtaining bioproducts from the studies of signals and interactions between microalgae and bacteria. Microorganisms, 10(10), 2029.
Hatti-Kaul, R., Chen, L., Dishisha, T., & Enshasy, H. E. (2018). Lactic acid bacteria: From starter cultures to producers of chemicals. FEMS Microbiology Letters, 365(20), fny213.
Doyle, L. M.; Wang, M. Z. Overview of Extracellular Vesicles, Their Origin, Composition, Purpose, and Methods for Exosome Isolation and Analysis. Cells 2019, 8, 727, doi:10.3390/cells8070727.
Yang, J.; Shin, T.-S.; Kim, J. S.; Jee, Y.-K.; Kim, Y.-K. A New Horizon of Precision Medicine: Combination of the Microbiome and Extracellular Vesicles. Exp Mol Med 2022, 54, 466-482, doi:10.1038/s12276-022-00748-6.
Chen, Y.; Li, G.; Liu, M.-L. Microvesicles as Emerging Biomarkers and Therapeutic Targets in Cardiometabolic Diseases. Genomics Proteomics Bioinformatics 2018, 16, 50-62, doi:10.1016fj.gpb.2017.03.006.
Di Daniele, A.; Antonucci, Y.; Campello, S. Migrasomes, New Vesicles as Hansel and Gretel White Pebbles? Biology Direct 2022, 17, 8, doi:10.1186/si3062-022-00321-1.
Zhang, H.; Freitas, D.; Kim, H. S.; Fabijanic, K.; Li, Z.; Chen, H.; Mark, M. T.; Molina, H.; Martin, A. B.; Bojmar, L.; et al. Identification of Distinct Nanoparticles and Subsets of Extracellular Vesicles by Asymmetric Flow Field-Flow Fractionation. Nat Cell Biol 2018, 20, 332-343, doi:10.1038/s41556-018-0040-4.
Fonseka, P.; Marzan, A. L.; Mathivanan, S. Introduction to the Community of Extracellular Vesicles. In New Frontiers: Extracellular Vesicles; Mathivanan, S., Fonseka, P., Nedeva, C., Atukorala, I., Eds.; Subcellular Biochemistry; Springer International Publishing: Cham, 2021; pp. 3-18 ISBN 978-3-030-67171-6.
Heijnen H F, Schiel A E, Fijnheer R, Geuze H J, Sixma J J. Activated platelets release two types of membrane vesicles: Microvesicles by surface shedding and exosomes derived from exocytosis of multivesicular bodies and alpha-granules. Blood. 1999; 94:3791-3799.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A composition for preventing Gram-negative bacteria growth comprising Ligilactobacillus sp. and/or bacterial extracellular vesicle isolated from Ligilactobacillus sp., wherein the Ligilactobacillus sp. is selected from the group consisting of Ligilactobacillus salivarius, and Ligilactobacillus saerimneri or combination thereof.

2. (canceled)

3. The composition of claim 1, wherein the Ligilactobacillus sp. is Ligilactobacillus salivarius UO.C109, Ligilactobacillus saerimneri UO.C121 and Ligilactobacillus salivarius UO.C249 or combination thereof.

4. (canceled)

5. The composition of claim 1, further comprising a second active ingredient, wherein the second active ingredient is a probiotic microorganism and/or a prebiotic and/or postbiotic.

6-7. (canceled)

8. The composition of claim 1, wherein the composition is formulated for oral administration or topical administration.

9. The composition of claim 1, formulated as a food additive, optionally wherein the food additive is an animal feed additive.

10. (canceled)

11. The composition of claim 9, wherein the animal feed additive is a livestock feed additive or aquaculture food additive.

12. The composition of claim 11, wherein the livestock feed additive is a cattle feed additive, equine feed additive, porcine feed additive or poultry feed additive.

13-15. (canceled)

16. A method of treating and/or preventing food poisoning and/or improving intestinal health and/or reducing bacterial pathogen load in a subject, the method comprising administering an effective amount of the composition of claim 1.

17. (canceled)

18. The method of claim 16, wherein the subject is a mammal, a fish, a bird, reptile or amphibian.

19. The method of claim 18, wherein the mammal is a human or domesticated animal; the bird is a poultry; and the fish is an aquaculture fish species.

20-21. (canceled)

22. A food product or animal feed comprising the composition of claim 1.

23. (canceled)

24. The animal feed of claim 22, wherein the animal feed is livestock feed or aquiculture feed.

25. The composition of claim 1 comprising the bacterial extracellular vesicles.

26-40. (canceled)

41. The method of claim 19, wherein the bird is a poultry.

42. The method of claim 41, wherein the composition is administered orally.

43. (canceled)

44. The method of claim 41, wherein the composition is administered within 24 hours of hatching.

45-47. (canceled)

48. The method of claim 19, wherein the domesticated animal is livestock.

49. The method of claim 48, wherein the composition is administered orally.

50-51. (canceled)

52. A method of inhibiting biofilm growth or reducing bacterial pathogen load on a surface, the method comprising treating a surface with the composition of claim 1.

53. (canceled)

54. The method of claim 52, wherein the method is to reduce contamination of eggs, vegetables or other food surfaces.

55. The method of claim 52, wherein the surface is a food preparation surface, food storage surface or a hard surface.


SEQUENCE LISTING (3 sequences)

SEQ ID NO: 1 - DNA, 1337 bp, Lactobacillus saerimneri
  source 1..1337: mol_type = genomic DNA
gcaagtcgagcgcatcggcccaactgattgaagatgcttgcatccgattgacgatggtttaccgatgagcggcggacgggtgagtaacacgtaggtaacctgcccagaagcgggggataacacctggaaacagatgctaataccgcataggtcatttgaccgcatggtcaaatgattaaagatggctctgctatcacttctggatggacctgcggcgtattagctagttggtaaggtaacggcttaccaaggcaatgatacgtagccgagttgagagactgatcggccacattgggactgagacacggcccagactcctacgggaggcagcagtagggaatcttccacaatggacgcaagtctgatggagcaacgccgcgtgagcgaagaaggtcttcggatcgtaaaactctgttgttagagaagaacacgggtgagagtaactgttcacctgttgacggtatctaaccagcaagtcacggctaactacgtgccagcagccgcggtaatacgtaggtggcaagcgttatccggatttattgggcgtaaagggaacgcaggcggttctttaagtctgatgtgaaagccttcggcttaaccgaagatgtgcattggaaactggggaacttgagtgcagaagaggagagtggaactccatgtgtagcggtgaaatgcgtagatatatggaagaacaccagtggcgaaagcggctctctggtctgtaactgacgctgaggttcgaaagcgtgggtagcgaacaggattagataccctggtagtccacgccgtaaacgatgaatgctaggtgttggagggtttccgcccttcagtgccgcagctaacgcactaagcattccgcctggggagtacgaccgcaaggttgaaactcaaaggaattgacgggggcccgcacaagcggtggagcatgtggtttaattcgaagctacgcgaagaaccttaccaggtcttgacatcttttgaccacctaagagattaggttttcccttcggggacaaaatgacaggtggtgcatggttgtcgtcagctcgtgtcgtgagatgttgggttaagtcccgcaacgagcgcaacccttgttgtcagttgccagcattcagttgggcactctggcgagactgccggtgacaaaccggaggaaggtggggatgacgtcaaatcatcatgccccttatgacctgggctacacacgtgctacaatgggcagtacaacgagtcgcgaaaccgcgaggtttagcaaatctcttaaagctgctctcagttcggactgcaggctgcaactcgcctgcacgaagtcggaatcgctagtaatcgcgaatcag

SEQ ID NO: 2 - DNA, 1313 bp, Ligilactobacillus salivarius
  source 1..1313: mol_type = genomic DNA
aacgaaactttcttacaccgaatgcttgcattcaccgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggcccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggttttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcgagaccgcgaggtttagctaatctcttaaagccgttctcagttcggattgtaggctgcaactcgcctacatgaagtcggaatcgctagtaatcgcgaa

SEQ ID NO: 3 - DNA, 1056 bp, Ligilactobacillus salivarius
  source 1..1056: mol_type = genomic DNA
tgctatacatgcaagtcgaacgaaactttcttacaccgaatgcttgcattcaccgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggcccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggctttcccttcggggacaaagtgacaggtggtgcatggctgtcgtcagctcgtgtcgtgag